(19) United States
(12) Patent Application Publication
(10) Pub. No.: US 2012/0060856 A1
(43) Pub. Date: Mar. 15, 2012

(54) Cosmetic Composition and Reshaping Method for Keratinic Fibers

(76) Inventors: Andreas Taden, Wittmund (DE); Hans G. Boerner, Potsdam (DE)

(21) Appl. No.: 13/299,429

(22) Filed: Nov. 18, 2011

Related U.S. Application Data
(63) Continuation of application No. PCT/EP2010/051372, filed on Feb. 4, 2010.

(30) Foreign Application Priority Data
May 20, 2009 (DE) 10 2009 003 291.6

(51) Int. Cl. A61K 8/64; A45D 7/04; A61K 8/41; A61K 8/49; A61K 8/44

(52) U.S. Cl. 132/203; 424/70.14; 424/70.1

(57) ABSTRACT

Cosmetic compositions containing, in a suitable carrier, at least one substance of general formula (I)

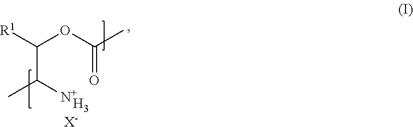

wherein R¹ is an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

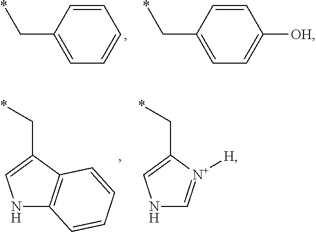

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

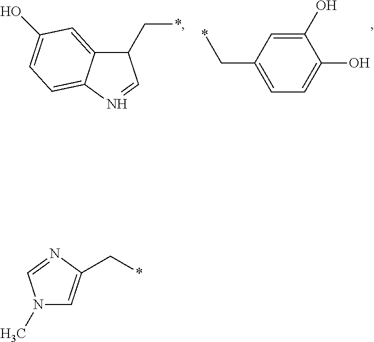

and X⁻ is a physiologically tolerable anion or the m^{th} part of an m-times charged ion, the compositions permitting more permanent styling without having to accept the disadvantages of a permanent waving process.

### CROSS REFERENCE TO RELATED APPLICATIONS

This application is a continuation of International Patent Application No. PCT/EP2010/051372 filed 4 Feb. 2010, which claims priority to German Patent Application No. 10 2009 003 291.6 filed 20 May 2009, both of which are incorporated herein by reference.

The present invention relates to a cosmetic composition and to a method for shaping keratinic fibers.

Lasting shaping of keratin fibers is typically performed by mechanically shaping the fibers and fixing the shape using suitable auxiliary agents. Before and/or after this shaping, the fibers are treated with an aqueous preparation of a keratin-reducing substance, and at the end of a contact period is rinsed out with water or an aqueous solution. In a second step, the fibers are then treated with an aqueous preparation of an oxidizing agent. At the end of a contact period this is also rinsed out and the fibers are freed from mechanical shaping aids (rollers, foam curlers). A known method of this type is the permanent waving of human hair. This method can be used both to create curls and waves in straight hair and to straighten curly hair.

Styling agents, which are usually preparation forms of film-forming polymers, are used for temporarily shaping keratinic fibers. These can be applied to the fibers as a spray, gel, powder, aqueous or alcoholic solution or dispersion, etc., and the hair then modeled into the desired shape with the hands or with combs and brushes.

The disadvantage of permanent waving is that the shape of the hairstyle cannot readily be changed. As its name suggests, this method of hair styling is designed to be relatively long-lasting. Moreover, a permanent wave treatment is time-consuming and should be carried out professionally (i.e., by a hairdresser). Another negative side-effect of permanent waving of hair in this way is an undesired “symmetrical” appearance of the hairstyle is often achieved. The curls are often very regular in shape and do not give the hairstyle the desired “youthful” appearance. Many user groups thus associate permanent waves with older people.

By contrast, conventional hair styling agents allow the shape of the hairstyle to be quickly altered. However, this is often accompanied by the disadvantage that the desired hairstyle shape has a limited lifetime. Depending on external parameters (humidity, wind, movement by the hairstyle wearer), conventional styling agents have to be applied several times a day in some cases in order to maintain the desired appearance. Conventional styling agents generally do not survive customary hygiene measures such as hair washing, requiring the hair to be restyled each time after showering or hair washing.

The present invention therefore attempts to improve the hair structure on a longer-lasting basis and allow a more permanent styling without having the disadvantages of a permanent wave process. At the same time, keratinic fibers damaged by external influences, such as coloring or previous bleaching or permanent wave processes, are ideally strengthened and repaired.

It has now been found that synthetic polymers containing a functional peptide block having a structure-changing unit (switch unit) can achieve this improvement. The switch unit bears a positive charge and can therefore interact particularly effectively with keratinic fibers. By changing the pH, an irreversible structural change can occur which changes a structural part of the polymers into protein structures. These are generally insoluble and exhibit a strong aggregation behavior. Very long-lasting and hair-repairing structures can be produced in this way.

The present invention firstly provides a cosmetic composition containing in a suitable carrier at least one substance having at least one structural unit of general formula (I)—

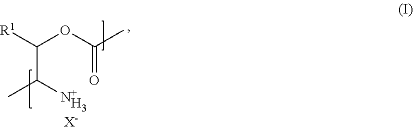

wherein R¹ is an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

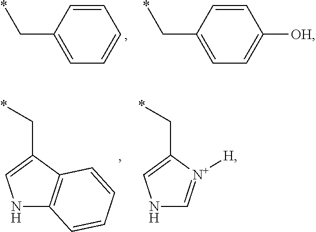

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

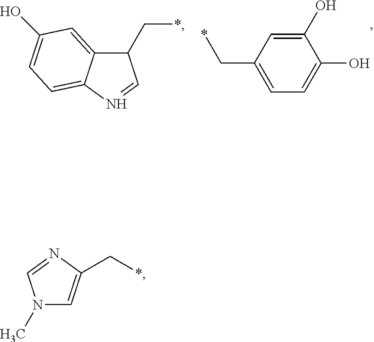

and
X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate.

Most particularly preferred compositions according to the invention contain in a suitable carrier at least one substance having at least one structural unit of general formula (I-1) to (I-29):

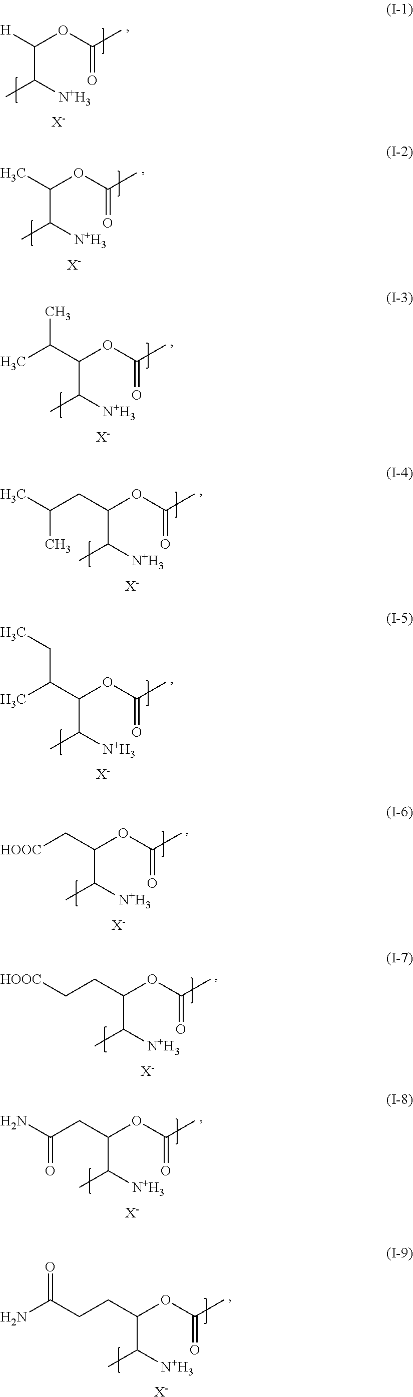

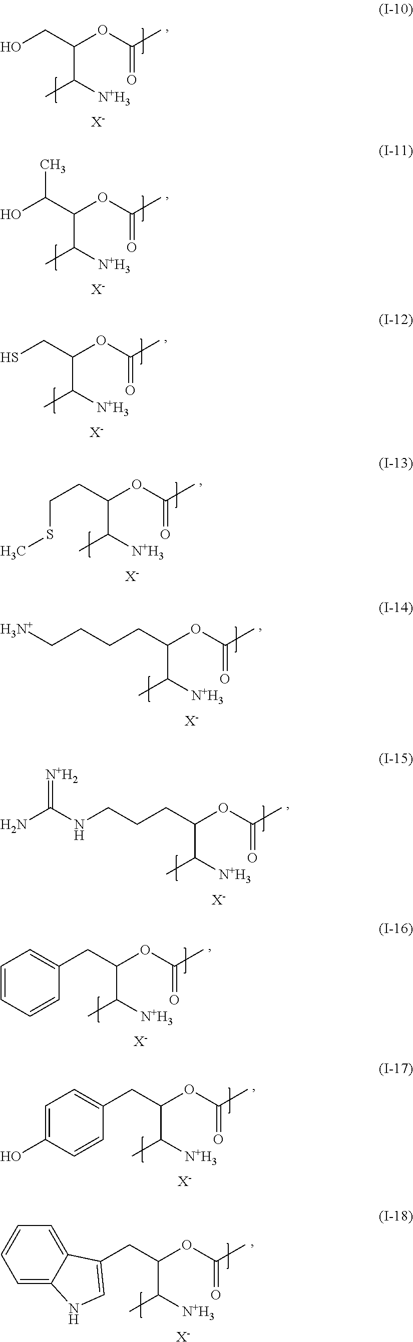

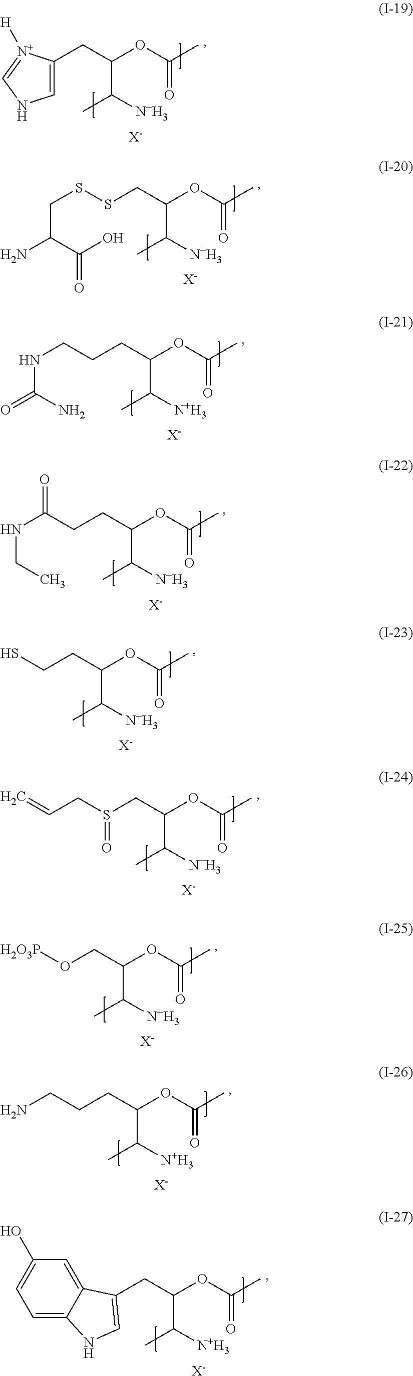

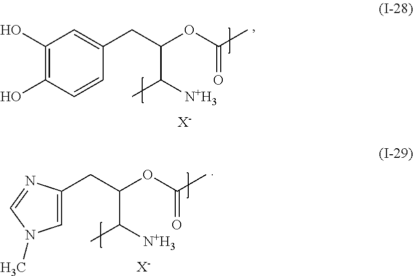

In formulae (I-1) to (I-29), X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate.

In the structural unit of general formula (I), an additional —C(O) grouping is preferably bonded to the ammonium group-bearing C atom. Most particularly preferred compositions according to the invention therefore contain at least one substance having at least one structural unit of general formula (I-a)—

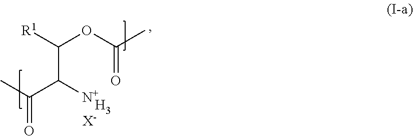

wherein R¹ and X⁻ are as defined above.

Most particularly preferred compositions of this embodiment contain in a suitable carrier at least one substance having at least one structural unit of general formulae (I-a-1) to (I-a-29):

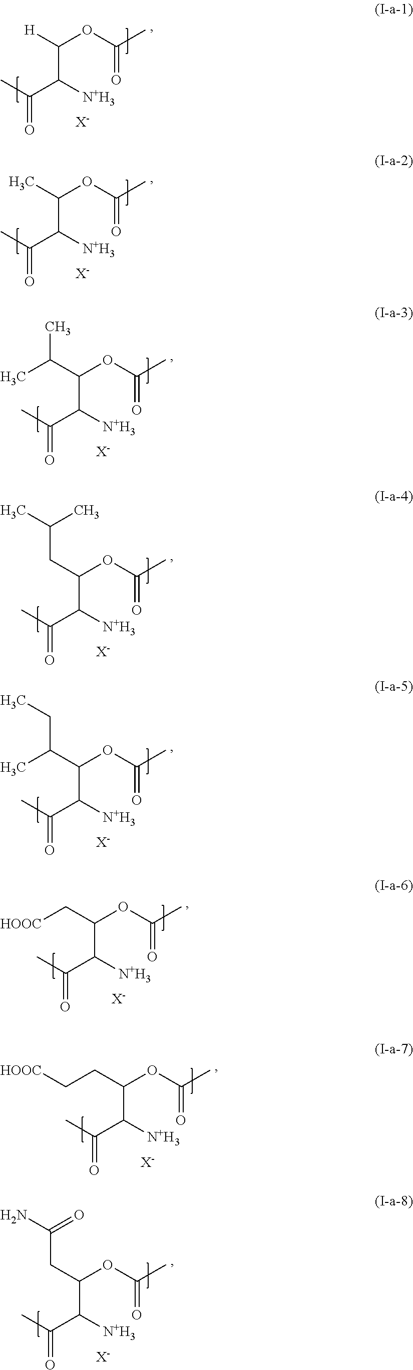

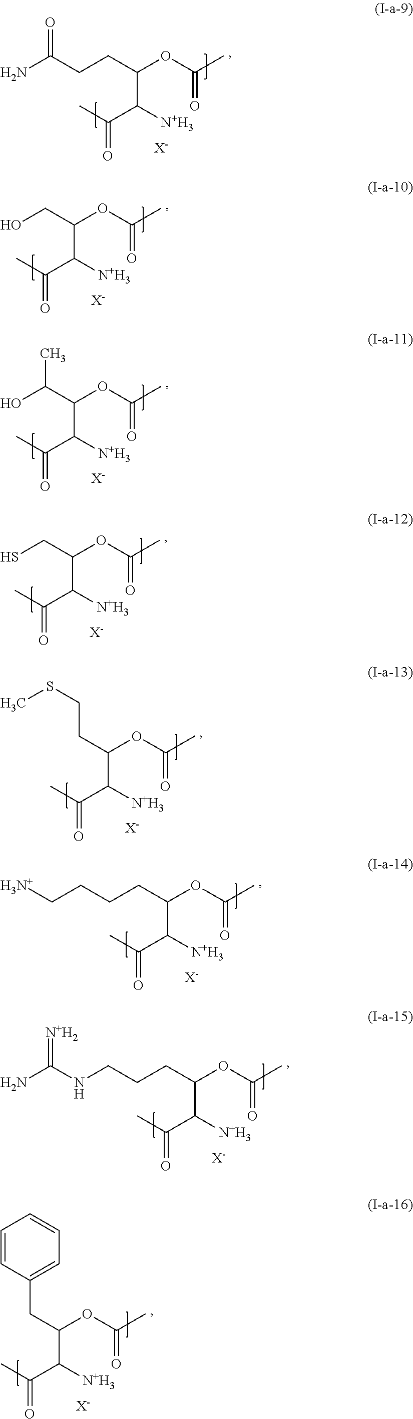

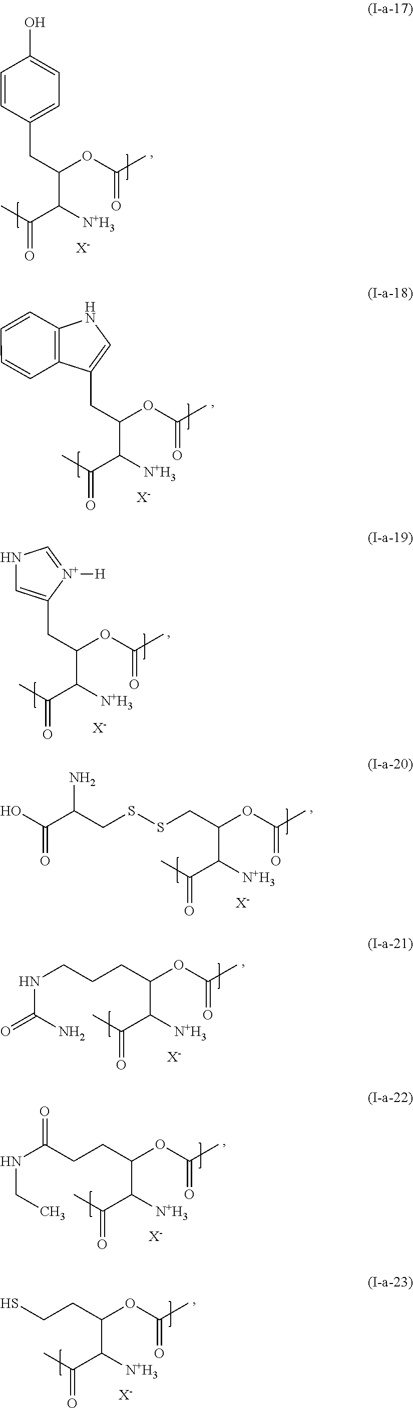

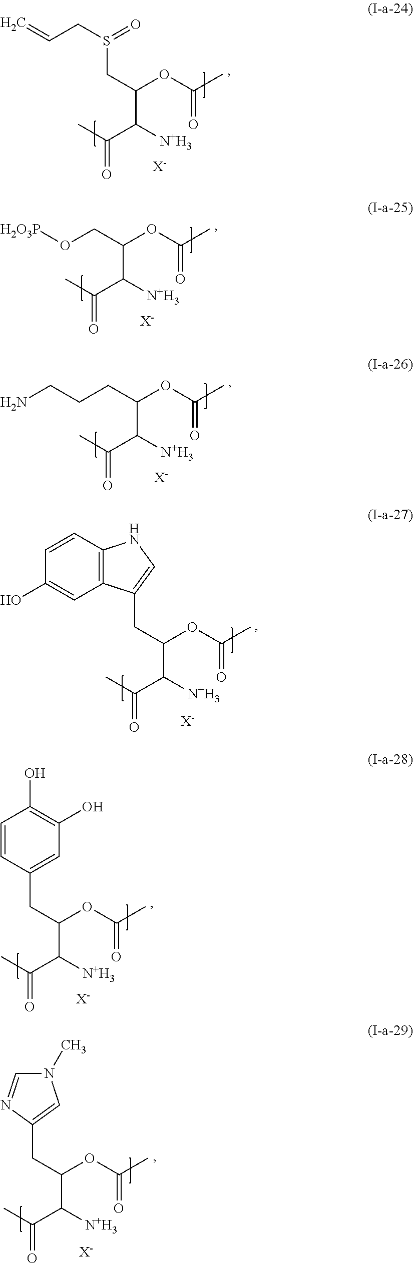

In formulae (I-a-1) to (I-a-29), X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate.

It is further preferable for an additional —CH(R²)— grouping to be bonded to the C atom of the —O—C(O) grouping in the structural unit of general formula (I) or (I-a). The R² group can be chosen from the same residues independently of the R¹ group.

Most particularly preferred compositions according to the invention therefore contain at least one substance having at least one structural unit of general formula (I-b)—

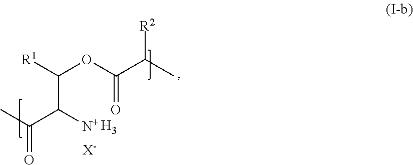

wherein R¹ and R² independently of each other are an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

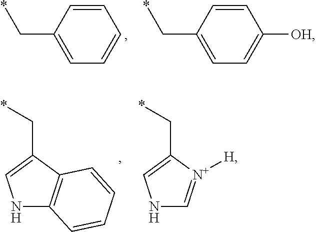

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

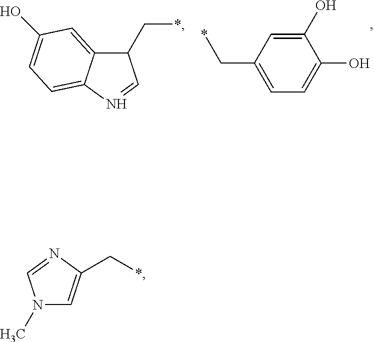

and
X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate.

Most particularly preferred compositions of this embodiment contain in a suitable carrier at least one substance having at least one structural unit of general formula (I-b-1) to (I-b-29):

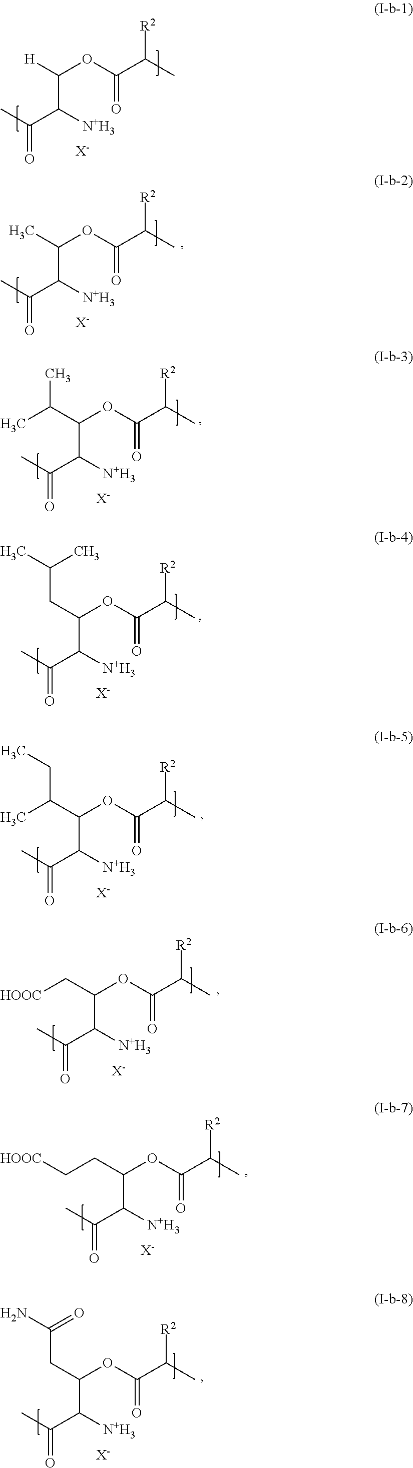

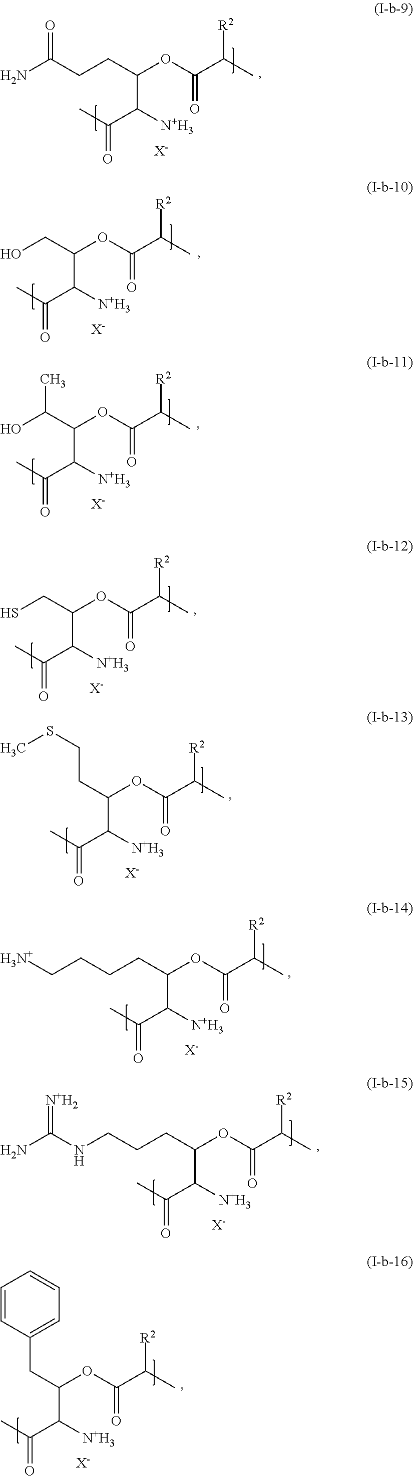

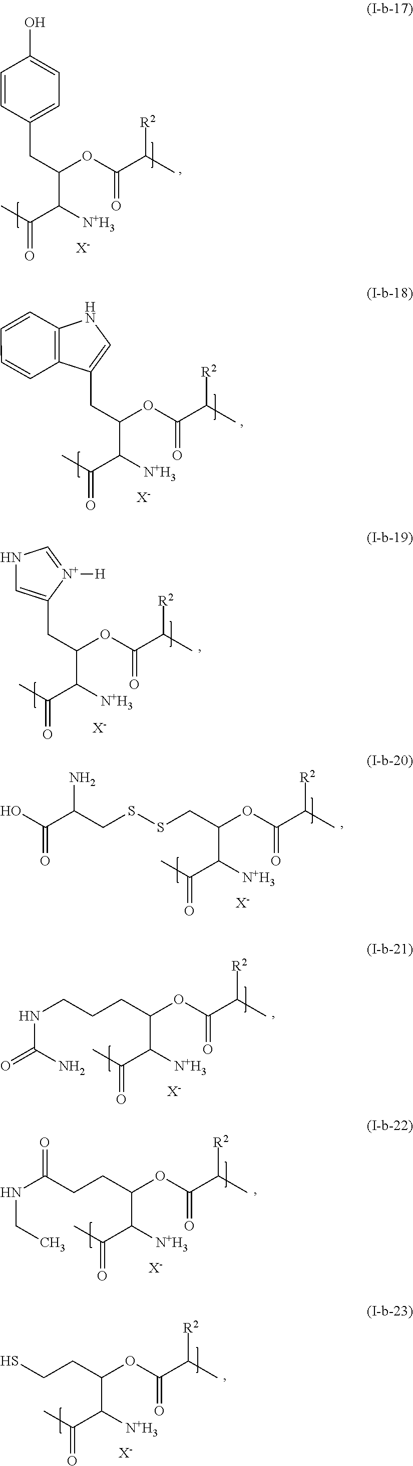

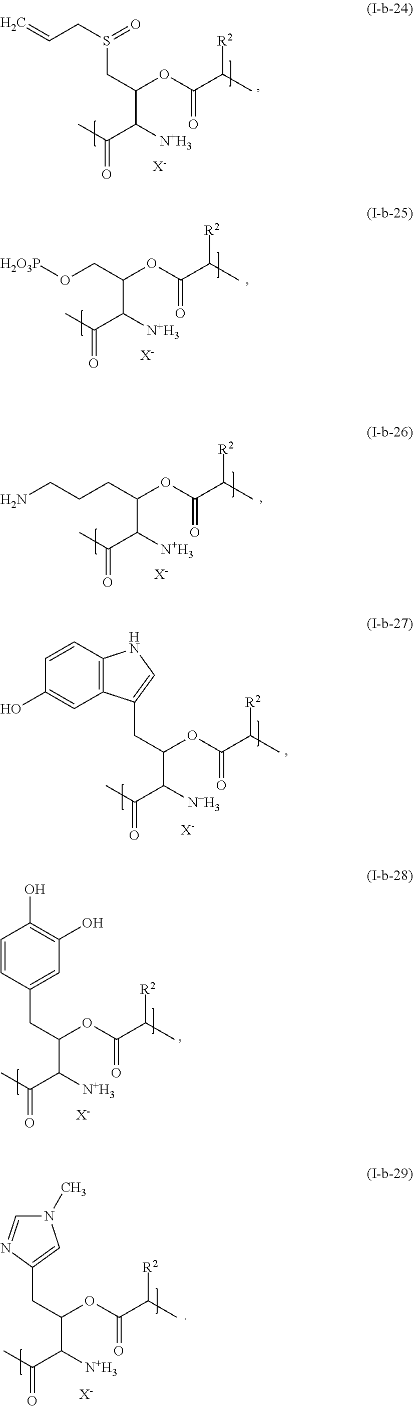

In formulae (I-b-1) to (I-b-29), X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate; and

R² is an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂NH₂),

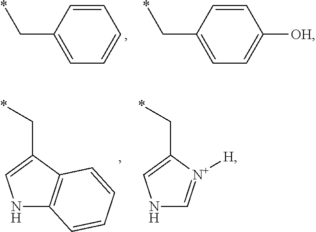

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

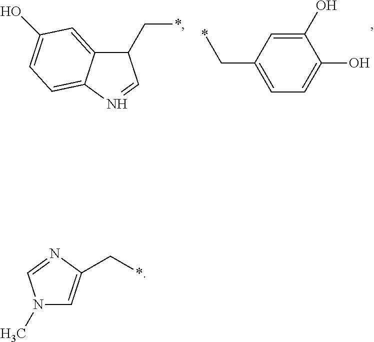

Of the aforementioned representatives, those of general formula (I-b-2) are most particularly preferred embodiments of the present invention.

It is still further preferable for an additional —CH(R²)—NH— grouping to be bonded to the C atom of the —O—C(O) grouping in the structural unit of general formula (I) or (I-a). This corresponds in formula (I-b) to an additional —NH— grouping on the C atom bearing the R² group.

The R² group can in turn be chosen from the same residues independently of the R¹ group. Most particularly preferred compositions according to the invention therefore contain at least one substance having at least one structural unit of general formula (I-c)—

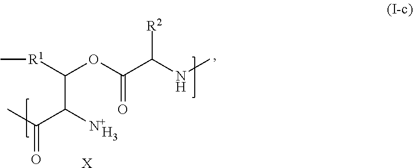

wherein R¹, R² and X⁻ are defined as above.

Most particularly preferred compositions of this embodiment contain in a suitable carrier at least one substance having at least one structural unit of general formula (I-c-1) to (I-c-29):

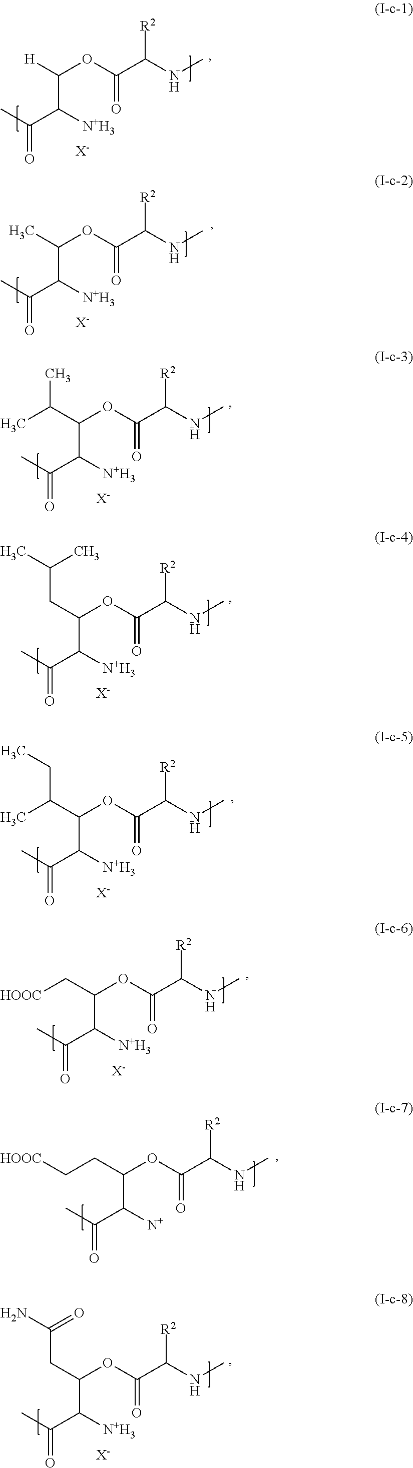

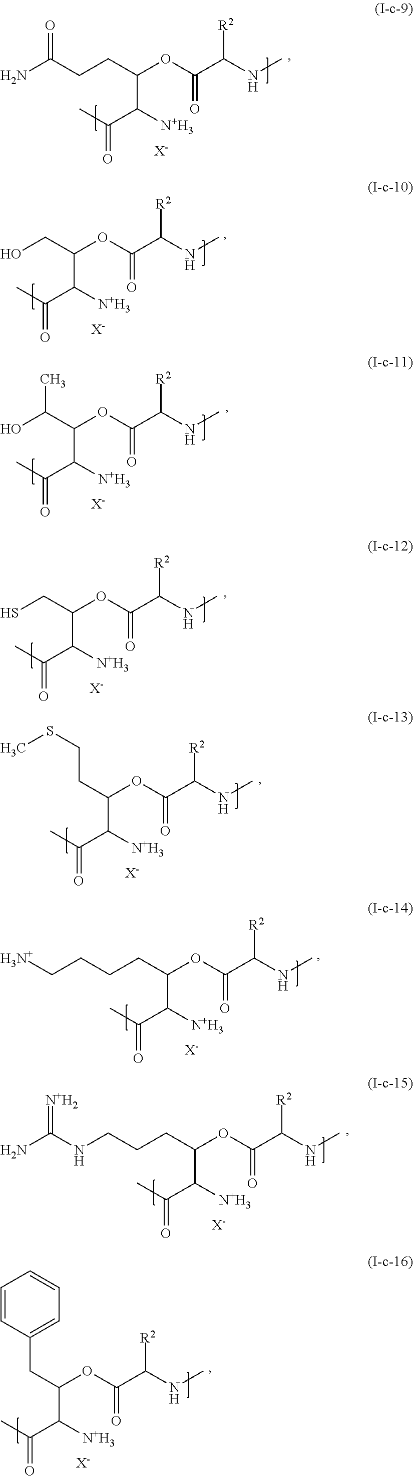

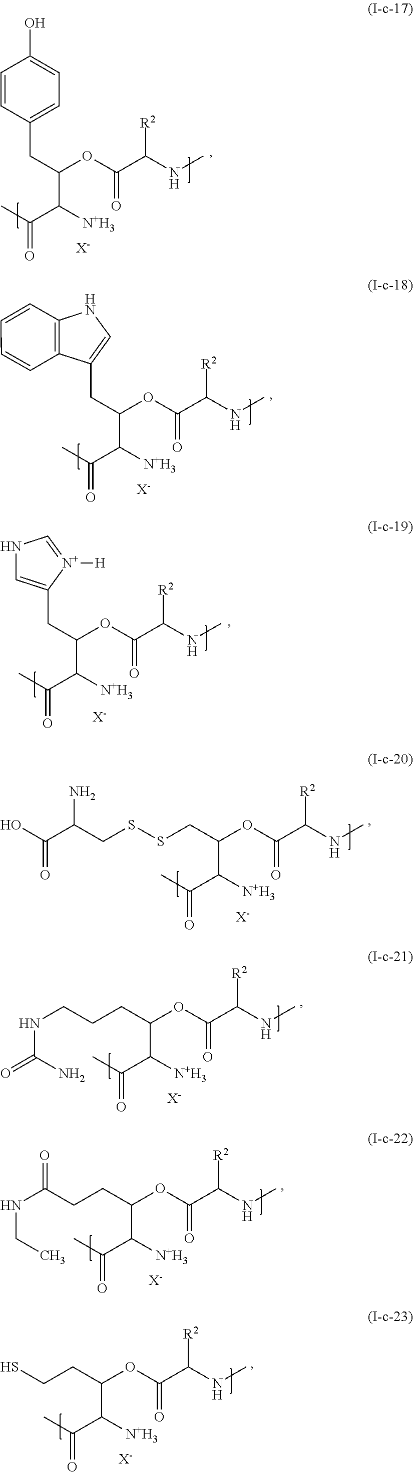

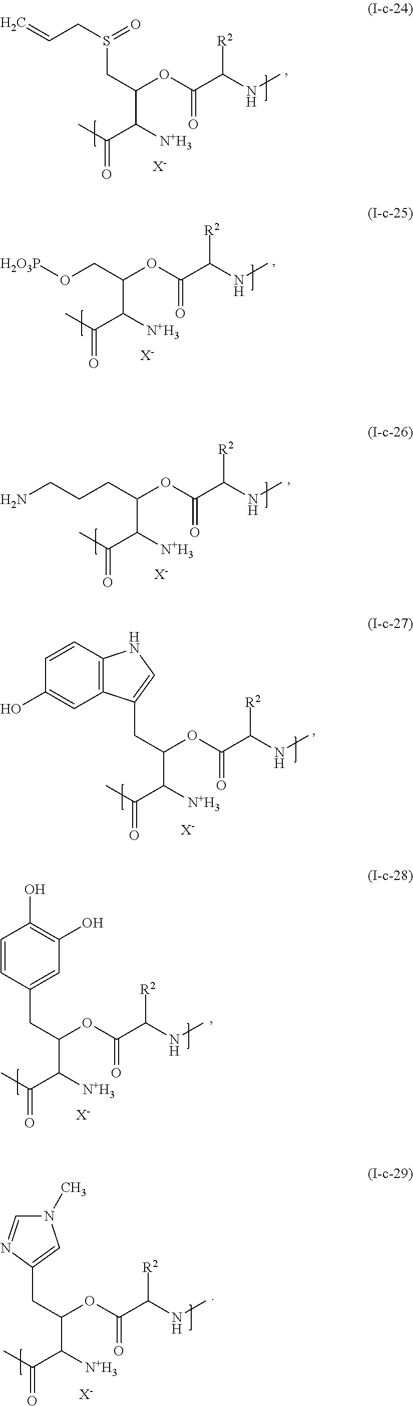

In formulae (I-c-1) to (I-c-29), X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate; and

R² is an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

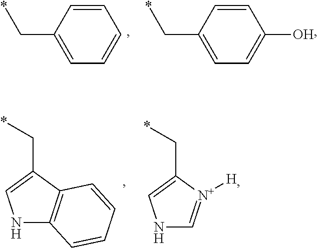

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

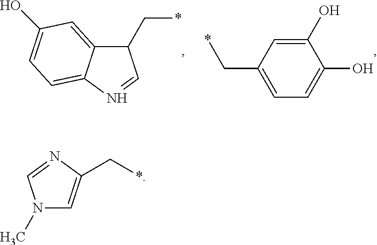

Of the aforementioned representatives, those of general formula (I-c-2) are most particularly preferred embodiments of the present invention.

Preferred compositions according to the invention contain at least one substance wherein structural units (I), (I-a), (I-b) or (I-c) are multiply bonded to one another. 2 to 200 structural units of formulae (I), (I-a), (I-b) or (I-c) can preferably follow on from one another. Substances having 2 to 90, preferably 2 to 80, more preferably 2 to 70, still more preferably 2 to 60 and in particular 2 to 50 repeating units (I), (I-a), (I-b) or (I-c) are most particularly preferred.

Most particularly preferred compositions contain structural unit (I-c) as a repeating unit. Preferred compositions according to the invention have at least one substance having at least one structural unit of general formula (I-d)—

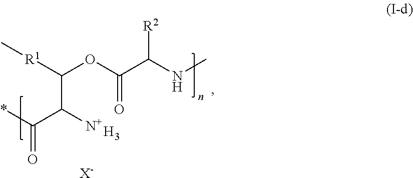

wherein R¹, R² and X⁻ are defined as above and n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. If n in formula (I-d) is 1, the result is formula (I-c).

Particularly preferred compositions according to the invention contain at least one substance having at least one structural unit of formulae (1) to (841) listed at paragraph [0025] in the priority document DE 10 2009 003 291 (A1). In formulae (1) to (841) listed therein, X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate, and n denotes values from 2 to 200, preferably 2 to 100, more preferably 2 to 50, and in particular 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40.

Most particularly preferred representatives of the aforementioned group are those of the formulae (30) to (59).

All groupings are suitable as the end group for substances having structural units of formulae (I), (I-a), (I-b), (I-c), (I-d) or (1) to (841) contained in compositions according to the invention, for example, —H, straight-chain or branched and substituted or unsubstituted alkyl residues, straight-chain or branched and substituted or unsubstituted alkenyl residues, straight-chain or branched and substituted or unsubstituted alkynyl residues, aryl residues, straight-chain or branched and substituted or unsubstituted aralkyl residues, groupings such as —NH₂, —OH, —NO₂, etc.

Preferred end groups according to the invention include—
—NH—CH₂—CH₂—OH,
—C(O)—CH(R³)—NH₂ with R³ chosen from the group from which R¹ and R² are also chosen,
—C(O)—CH(R³)—NH—C(O)—CH₂CH₂—C(O)—NH-(EO)ₙ—H with n=1 to 200,
—C(O)—CH(R³)—NH—C(O)—CH₂CH₂—C(O)—NH—(PO)ₙ—H with n=1 to 200, and
—C(O)—CH(R³)—NH—C(O)—CH₂CH₂—C(O)—NH-(EO)ₙ(PO)ₘH with n+m=1 to 200.

Most particularly preferred compositions according to the invention contain at least one substance of formula (II)—

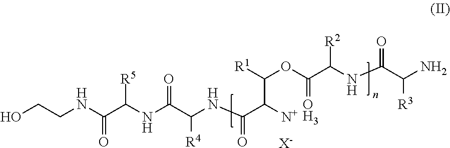

wherein R¹, R², n and X⁻ are defined as above, and R³, R⁴ and R⁵ are independently chosen from

### —H, —CH₃, —CH(CH₃)₂, —CH₂CH(CH₃)₂, —CH(CH₃)CH₂CH₃,

—CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

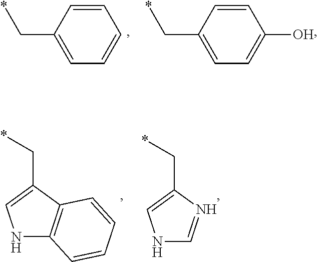

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

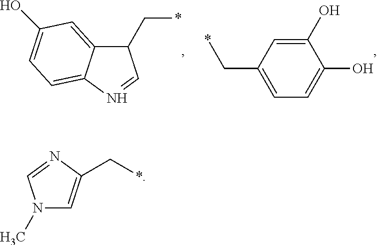

Of the alkoxylated representatives of compounds containing structural units of formulae (I), (I-a), (I-b), (I-c), (I-d), or (1) to (841), ethoxylated and/or propoxylated compounds are particularly preferred. Most particularly preferred compositions contain at least one substance of formula (III)—

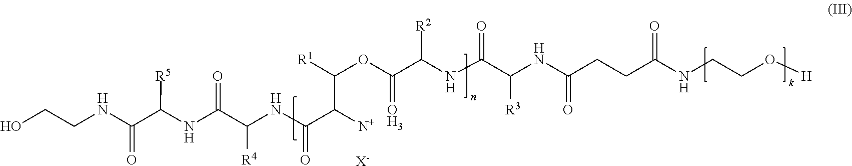

R¹, R², n and X⁻ are defined as above, and R³, R⁴ and R⁵ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

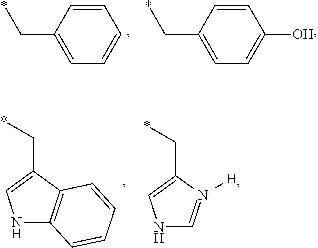

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

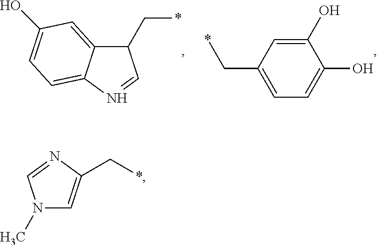

and
k is a value from 1 to 100, preferably 1, 2, 3, 4, 5, 6, 7, 8, 9 or 10.

Likewise particularly preferred are compositions according to the invention containing at least one substance of formula (IV)—

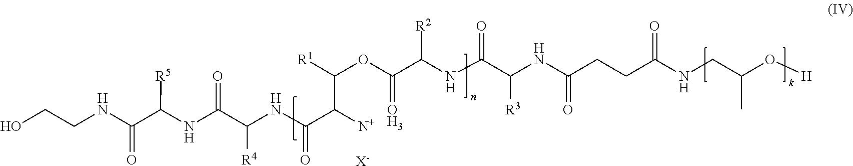

wherein R¹, R², n and X⁻ are defined as above, and R³, R⁴ and R⁵ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

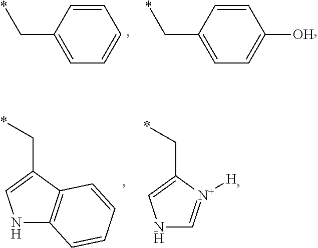

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

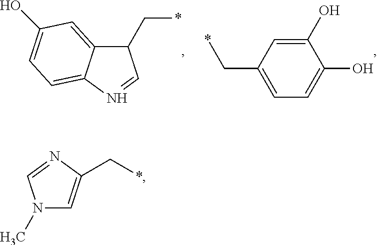

and
k is a value from 1 to 50, preferably 1, 2, 3, 4, 5, 6, 7, 8, 9 or 10.

EO or PO units or mixed alkoxylated groups can also be bonded to the other end of the molecule. Compositions according to the invention preferred here contain at least one substance of formula (VI)—

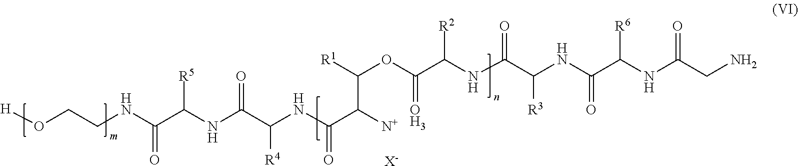

wherein R¹, R², n and X⁻ are defined as above, and R³, R⁴, R⁵ and R⁶ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂),

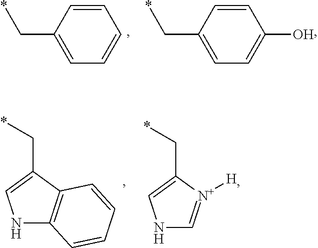

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

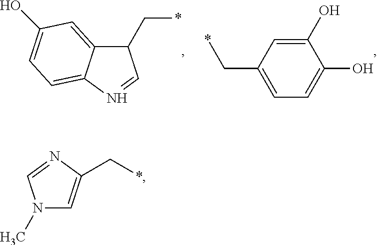

and
m is a value from 1 to 50, preferably 1, 2, 3, 4, 5, 6, 7, 8, 9 or 10.

Likewise preferred are compositions according to the invention containing at least one substance of formula (VI)—

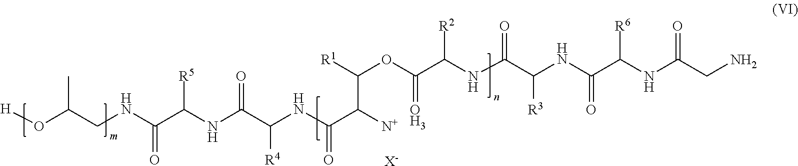

wherein R¹, R², n and X⁻ are defined as above, and R³, R⁴, R⁵ and R⁶ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄N⁺H₃, —(CH₂)₃—NH—C═N⁺H₂(NH₂),

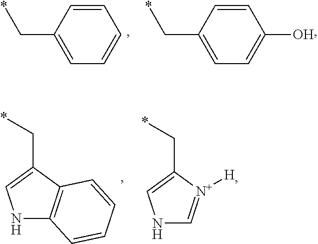

—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

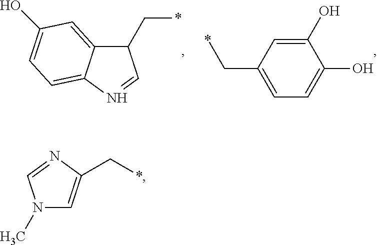

and
m is a value from 1 to 50, preferably 1, 2, 3, 4, 5, 6, 7, 8, 9 or 10.

Regardless of which specific compounds having structural units of formulae (I), (I-a), (I-b), (I-c), (I-d) or (1) to (841) are present in the agents, the agents preferably have molar masses of from 250 to 100,000 Daltons. Preferred compositions according to the invention are those wherein the substance(s) containing at least one structural unit of general formula (I) as defined above have molar masses of 250 to 100,000 gmol⁻¹, preferably 500 to 50,000 gmol⁻¹, more preferably 750 to 25,000 gmol⁻¹ and in particular 1000 to 10,000 gmol⁻¹.

Likewise, regardless of which specific compounds having structural units of formulae (I), (I-a), (I-b), (I-c), (I-d) or (1) to (841) are present in the agents and irrespective of their molar mass, compounds having structural units of formulae (I), (I-a), (I-b), (I-c), (I-d) or (1) to (841) are preferably present in the agents in defined amounts. Preferred compositions according to the invention contain, relative to their weight, 0.001 to 10 wt. %, preferably 0.0025 to 7.5 wt. %, more preferably 0.005 to 5 wt. %, particularly preferably 0.01 to 4 wt. %, more preferably 0.05 to 3 wt. % and in particular 0.1 to 3 wt. % of substance(s) having at least one structural unit of general formula (I) as defined above.

Particularly preferred compositions according to the invention have a weakly acid to acid pH. Compositions are most particularly preferred which have a pH of 6.5 or lower, preferably 6 or lower, more preferably 5.5 or lower and in particular 5 or lower.

These pH values can be established, for example, with appropriate acids such as hydrochloric acid, hydrobromic acid, hydroiodic acid, sulfuric acid, citric acid, phosphoric acid, methylsulfuric acid or p-toluenesulfonic acid. Use of a buffer system is most particularly preferred according to the invention, wherein citric acid/citrate buffers and phosphoric acid/phosphate buffers have proved to be outstandingly suitable.

At low pH values, compounds having structural units of formulae (I), (I-a), (I-b), (I-c), (I-d), or (1) to (841) present in the compositions have an ammonium group:

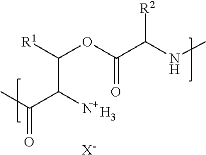

At higher pH values this is deprotonated:

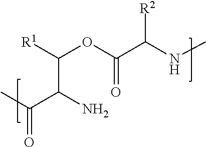

and rearranges itself irreversibly by O->N acyl transfer:

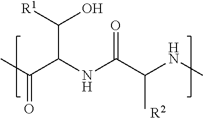

The peptide-like structure obtained in this way has a high affinity for keratinic fibers. The rearranged molecules adhere very well and give the hair stability, elasticity and hold. More lasting styling shapes can be achieved in this way, and previously damaged hair is repaired.

Compositions according to the invention can contain further conventional cosmetics ingredients, particularly further styling agent ingredients. Compounds having structural units of formulae (I), (I-a), (I-b), (I-c), (I-d) or (1) to (841) therein are advantageously supplemented by a content of further polymers in the agents.

Particularly advantageous polymers useful in compositions according to the invention are described below.

Preferred compositions according to the invention contain at least one copolymer A having—
at least one structural unit according to formula (A-I)

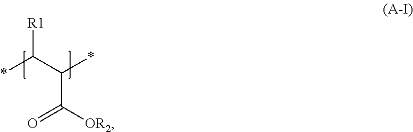

wherein R1 is —H or —CH₃ and R2 is —H or —CH₃ or —CH₂CH₃ or —CH₂CH₂CH₃ or —CH(CH₃)₂,
and at least one further structural unit different from structural unit (A-I) according to formula (A-II)

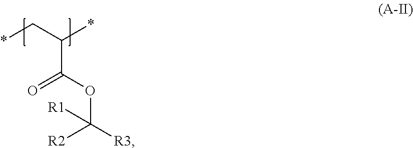

wherein R1 and R2 are independently —CH₃ or —CH₂CH₃ or —CH₂CH₂CH₃ or —CH(CH₃)₂ and R3 is a saturated or unsaturated, straight-chain or branched hydrocarbon residue.

Compositions of this preferred embodiment contain a polymer made up of at least two different monomers of formulae (A-I) and (A-II). Further monomers can moreover be incorporated by polymerization.

The first monomer present in copolymer A can be described by formula (A-I) (see above), wherein R1 is —H or —CH₃ and R2 is —H or —CH₃ or —CH₂CH₃ or —CH₂CH₂CH₃ or —CH(CH₃)₂. If R1 is —H, the monomers of formula (I) are acrylic acid or acrylic acid ester. If R1=—CH₃, the monomers of formula (I) are methacrylic acid or methacrylic acid ester.

Most particularly preferred monomers of formula (A-I) are acrylic acid, methacrylic acid, methyl acrylate, methyl methacrylate, ethyl acrylate, ethyl methacrylate, propyl acrylate, propyl methacrylate, isopropyl acrylate and isopropyl methacrylate.

Particularly preferred compositions according to the invention contain acrylic acid or acrylic acid ester as the monomer unit in copolymer A. Such compositions are those wherein copolymer A contains structural units of formula (A-Ia)—

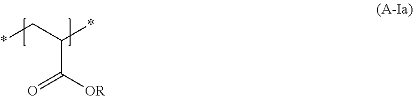

wherein R is —H or —CH₃ or —CH₂CH₃ or —CH₂CH₂CH₃ or —CH(CH₃)₂.

The second monomer present in copolymer A can be described by formula (A-II) (see above) wherein R1 and R2 are independently —CH₃ or —CH₂CH₃ or —CH₂CH₂CH₃ or —CH(CH₃)₂ and R3 is a saturated or unsaturated, straight-chain or branched hydrocarbon residue.

Of the representatives of formula (A-II), monomers are preferred in which R1=R2. Particularly preferred monomers can be described by formulae (A-IIa), (A-IIb) and (A-IIc):

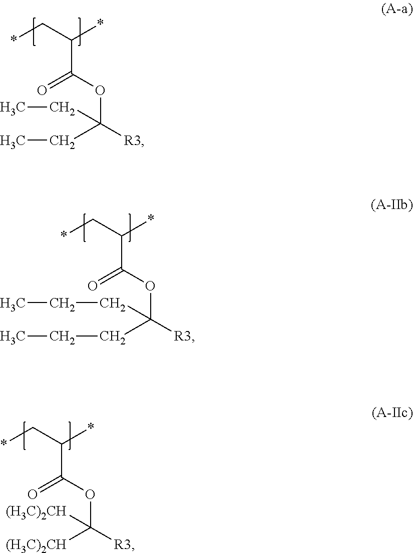

wherein R3 is a saturated or unsaturated, straight-chain or branched hydrocarbon residue.

Most particularly preferred monomers are those in which R1=R2=—CH₃. It is further preferable to choose a straight-chain alkyl residue as R3, with ethyl, n-butyl, n-hexyl, n-octyl and n-decyl residues being preferred.

Most particularly preferred compositions according to the invention are those wherein copolymer A contains structural units of formula (A-IIa)—

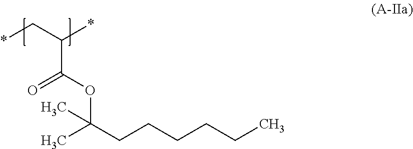

Compositions according to the invention are still more preferred which contain copolymer(s) (A) with molar masses of 10 to 750 kDa, preferably 25 to 500 kDa, more preferably 30 to 400 kDa and in particular 4 to 250 kDa.

Regardless of the nature and molar mass of copolymers A used, preferred compositions according to the invention contain, based on total weight of the ready-to-use compositions, 0.1 to 10 wt. %, preferably 0.5 to 7.5 wt. % and in particular 1 to 5 wt. % of copolymer(s) A.

Monomers of formulae (A-I) and (A-II) are preferably present in copolymer A within defined limits. Preferred compositions contain copolymer(s) A having—
10 to 95 mol %, preferably 15 to 85 mol % and in particular 20 to 80 mol % of monomers of formula (A-I),
5 to 90 mol %, preferably 7.5 to 80 mol % and in particular 10 to 60 mol % of monomers of formula (A-II).

Particularly preferred compositions according to the invention contain at least one further copolymer B having—
at least one structural unit according to formula (B-I)

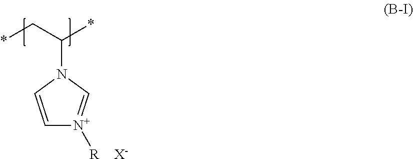

wherein R is a C₁ to C₃₀ alkyl group, a C₁ to C₄ aralkyl group, a C₂ to C₆ alkenyl group or a C₂ to C₆ hydroxyalkyl group; and X⁻ is a physiologically tolerable anion;
and at least one further structural unit according to formula (B-II)

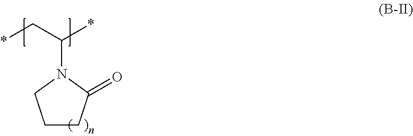

wherein n is 1, 2 or 3 as the number of methylene units.

Film-forming and/or fixing copolymers B are known. These copolymers have at least one structural unit according to formula (B-I) and at least one structural unit according to formula (B-II) and can moreover have further structural units incorporated by addition of corresponding monomers during polymerization.

In formula (B-I), R is a C₁ to C₃₀ alkyl group, a C₁ to C₄ aralkyl group, a C₂ to C₆ alkenyl group or a C₂ to C₆ hydroxyalkyl group. Preferred R groups include —CH₃, —CH₂CH₃, —CH₂CH₂CH₃, CH(CH₃)₂, —(CH₂)₃CH₃, —CH₂—CH(CH₃)₂, CH(CH₃)CH₂CH₃, —C(CH₃)₃, —CH₂OH, —CH₂CH₂OH, —CH₂CH₂CH₂OH, —CH(OH)CH₂CH₃, —CH₂CH(OH)CH₃.

X⁻ is a physiologically tolerable anion. Preferred anions are chloride, bromide, iodide, sulfate, methosulfate, ethyl sulfate, tosylate and tetrafluoroborate.

In formula (B-II), n is the number of methylene groups. If n=1, formula (B-II) is a vinyl pyrrolidone unit; if n=2, it is a vinyl piperidinone unit; and if n=3, it is a vinyl caprolactam unit.

Particularly preferred compositions according to the invention have as copolymer B a copolymer B1 having—
at least one structural unit according to formula (B-I) (see above) wherein R is a methyl group and X is methosulfate,
at least one further structural unit according to formula (B-II) (see above) wherein n is 1 methylene unit.

Most particularly preferred copolymers B1 contain 10 to 30 mol %, preferably 15 to 25 mol % and in particular 20 mol % of structural units according to formula (B-I) and 70 to 90 mol %, preferably 75 to 85 mol % and in particular 80 mol % of structural units according to formula (B-II).

It is particularly preferable here for copolymers B1 to contain, in addition to polymer units resulting from the incorporation of the cited structural units according to formula (B-I) and (B-II) in the copolymer, a maximum of 5 wt. %, preferably a maximum of 1 wt. % of polymer units resulting from the incorporation of other monomers. Copolymers B1 are preferably made up exclusively of structural units of formula (B-I) and (B-II) and can be described by the general formula—

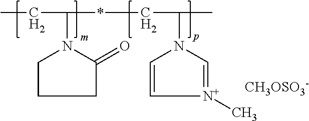

wherein m and n vary according to the molar mass of the polymer and are not intended to suggest that these are block copolymers. Structural units of formula (B-I) and formula (B-II) can rather be randomly distributed in the molecule.

Such N-methyl vinyl imidazole/vinyl pyrrolidone copolymers are referred to under INCI nomenclature as POLYQUATERNIUM-44 and are available, for example, from BASF under the trade name Luviquat® UltraCare.

Particularly preferred compositions according to the invention contain a copolymer B1 having molar masses within a defined range. Preferred compositions according to the invention are those wherein copolymer B1 has a molar mass of 50 to 400 kDa, preferably 100 to 300 kDa, more preferably 150 to 250 kDa and in particular 190 to 210 kDa.

In addition to copolymer(s) B1 or in its or their place, compositions according to the invention can also contain copolymers B2, which as additional structural units contain structural units of formula (B-II), wherein n is the number 3.

Further particularly preferred compositions according to the invention have as copolymer B a copolymer B2, containing—
at least one structural unit according to formula (B-I) (see above), wherein R is a methyl group and X is methosulfate,
at least one further structural unit according to formula (B-II) (see above), wherein n is 1 methylene unit, and
at least one further structural unit according to formula (B-II) (see above), wherein n is 3 methylene units.

Here too it is particularly preferable for copolymers B2 to contain, in addition to polymer units resulting from the incorporation of cited structural units according to formula (B-I) and (B-II) in the copolymer, a maximum of 5 wt. %, preferably a maximum of 1 wt. %, of polymer units resulting from the incorporation of other monomers. Copolymers B2 are preferably made up exclusively of structural units of formula (B-I) and (B-II) and can be described by the general formula—

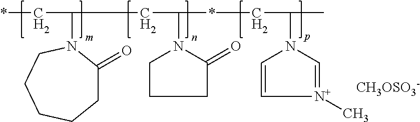

wherein m, n and p vary according to the molar mass of the polymer and are not intended to suggest that these are block copolymers. Structural units of formula (B-I) and formula (B-II) can rather be randomly distributed in the molecule.

Such N-methyl vinyl imidazole/vinyl pyrrolidone/vinyl caprolactam copolymers are referred to under INCI nomenclature as POLYQUATERNIUM-46 and are available for example from BASF under the trade name Luviquat® Hold.

Most particularly preferred copolymers B2 contain 1 to 20 mol. %, preferably 5 to 15 mol. % and in particular 10 mol % of structural units according to formula (B-I) and 30 to 50 mol %, preferably 35 to 45 mol % and in particular 40 mol % of structural units according to formula (B-II) with n=1 and 40 to 60 mol %, preferably 45 to 55 mol % and in particular 60 mol % of structural units according to formula (B-II) with n=3.

Particularly preferred compositions according to the invention contain a copolymer B2 having molar masses within a defined range. Compositions according to the invention are preferred here in which the copolymer B2 has a molar mass of 100 to 1000 kDa, preferably 250 to 900 kDa, more preferably 500 to 850 kDa and in particular 650 to 710 kDa.

In addition to copolymer(s) B1 and/or B2 or in its or their place, compositions according to the invention can also contain copolymers B3, which as additional structural units contain structural units of formula (B-II), wherein n is the number 3, and further distructural units from the group of vinyl imidazole units and further structural units from the group of acrylamide and/or methacrylamide units.

Further particularly preferred compositions according to the invention have as copolymer B a copolymer B3 having—
at least one structural unit according to formula (B-I) (see above), wherein R is a methyl group and X is methosulfate,
at least one further structural unit according to formula (B-II) (see above), wherein n is 1 methylene unit,
at least one further structural unit according to formula (B-III)

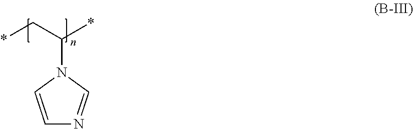

at least one further structural unit according to formula (B-IV)

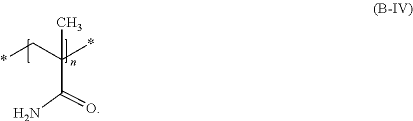

Here too it is particularly preferable for copolymers B3 to contain, in addition to polymer units resulting from the incorporation of the cited structural units according to formula (B-I), (B-II), (B-III) and (B-IV) in the copolymer, a maximum of 5 wt. %, preferably a maximum of 1 wt. %, of polymer units resulting from the incorporation of other monomers. Copolymers B3 are preferably exclusively made up of structural units of formula (B-I), (B-II), (B-III) and (B-IV) and can be described by the general formula—

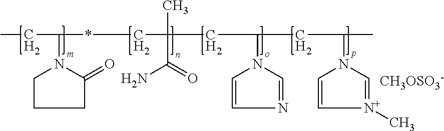

wherein m, n, o and p vary according to the molar mass of the polymer and are not intended to suggest that these are block copolymers. Structural units of formulae (B-I), (B-II), (B-III) and (B-IVI) can rather be randomly distributed in the molecule.

Such N-methyl vinyl imidazole/vinyl pyrrolidone/vinyl imidazole/methacrylamide copolymers are referred to under INCI nomenclature as POLYQUATERNIUM-68 and are available, for example, from BASF under the trade name Luviquat® Supreme.

Most particularly preferred copolymers B3 contain 1 to 12 mol. %, preferably 3 to 9 mol. % and in particular 6 mol % of structural units according to formula (B-I), and 45 to 65 mol %, preferably 50 to 60 mol % and in particular 55 mol % of structural units according to formula (B-II) with n=1, and 1 to 20 mol %, preferably 5 to 15 mol % and in particular 10 mol % of structural units according to formula (B-III), and 20 to 40 mol %, preferably 25 to 35 mol % and in particular 29 mol % of structural units according to formula (B-IV).

Particularly preferred compositions according to the invention contain a copolymer B3 having molar masses within a defined range. Preferred compositions according to the invention are those wherein copolymer B3 has a molar mass of 100 to 500 kDa, preferably 150 to 400 kDa, more preferably 250 to 350 kDa and in particular 290 to 310 kDa.

Regardless of whether just one copolymer B or several copolymers B are used and irrespective of the choice of specific copolymer B, preferred compositions are those wherein the total amount of copolymers B, based on total weight of the ready-to-use compositions, is 0.05 to 5 wt. %, preferably 0.1 to 4 wt. % and in particular 0.25 to 3 wt. %.

In addition to film-forming copolymers B or in their place, compositions according to the invention can also contain film-forming polymers C from the group of acrylate polymers (i.e., polymers containing at least one monomer unit from the acrylic acid and/or methacrylic acid group and/or esters thereof). Preferred compositions according to the invention contain at least one acrylate polymer C chosen from—
c1) polyacrylic acid and/or
c2) copolymers of methacrylic acid with acrylamidopropane sulfonic acid and/or
c3) copolymers of acrylic acid with methacrylic acid and acrylic acid esters and/or
c4) copolymers of acrylic acid with methacrylic acid with acrylic acid esters and methacrylic acid esters and/or
c5) copolymers of acrylic acid esters with methacrylic acid.

Thus, preferred compositions according to the invention contain, for example, polyacrylic acid as the polymer C. This has structural units of the formula—

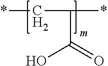

wherein m varies according to the molar mass.

Particularly preferred compositions according to the invention contain as polymer c1 polyacrylic acids having a molar mass of 10 to 250 kDa, preferably 25 to 200 kDa, more preferably 50 to 150 kDa and in particular 70 to 100 kDa.

Polymers c1 are preferably used within defined quantity ranges. Preferred compositions contain, relative to the weight of the ready-to-use compositions, 0.05 to 5 wt. %, preferably 0.1 to 4 wt. % and in particular 0.25 to 3 wt. % of polymer(s) c1.

In addition to polymer(s) c1 or in its or their place, compositions according to the invention can also contain polymers c2 from the group of copolymers of methacrylic acid with acrylamidopropane sulfonic acid.

These can be described by the general formula—

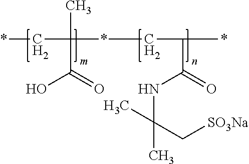

wherein m and n vary according to the molar mass of the polymer and are not intended to suggest that these are block copolymers. The structural units can rather be randomly distributed in the molecule.

Particularly preferred compositions according to the invention contain as copolymer c2 copolymers of methacrylic acid with acrylamidopropane sulfonic acid having a molar mass of 100 to 2500 kDa, preferably 250 to 2000 kDa, more preferably 500 to 1750 kDa and in particular 800 to 1500 kDa.

Copolymers c2 are preferably used within defined quantity ranges. Preferred compositions contain, based on total weight of the ready-to-use compositions, 0.05 to 5 wt. %, preferably 0.1 to 4 wt. % and in particular 0.25 to 3 wt. % of copolymer(s) c2.

Copolymers of methacrylic acid and acrylamidopropane sulfonic acid are available, for example, under the trade name Fixomer® A-30 (Nalco).

In addition to the polymer(s) c1 and/or copolymer(s) c2 or in its or their place, compositions according to the invention can also contain polymers c3 from the group of copolymers of acrylic acid with methacrylic acid and acrylic acid esters.

These can be described by the general formula—

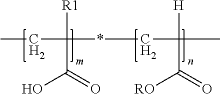

wherein m and n vary according to the molar mass of the polymer and are not intended to suggest that these are block copolymers. The structural units can rather be randomly distributed in the molecule. R1 denotes —H or —CH₃.

Particularly preferred compositions according to the invention contain as copolymer c3 copolymers of acrylic acid with methacrylic acid and acrylic acid esters having a molar mass of 50 to 500 kDa, preferably 100 to 400 kDa, more preferably 150 to 300 kDa and in particular 200 to 250 kDa.

Copolymers c3 are preferably used within defined quantity ranges. Preferred compositions contain, based on total weight of the ready-to-use compositions, 0.05 to 5 wt. %, preferably 0.1 to 4 wt. % and in particular 0.25 to 3 wt. % of copolymer(s) c3.

A most particularly preferred copolymer c3 is referred to under INCI nomenclature as Acrylates Copolymer. Such a polymer is available for example under the trade name Aculyn® 33A (Rohm & Haas).

In addition to the polymer(s) c1 and/or copolymer(s) c2 and/or copolymer(s) c3 or in its or their place, compositions according to the invention can also contain polymers c4 from the group of copolymers of acrylic acid with methacrylic acid and ethoxylated acrylic acid esters and ethoxylated methacrylic acid esters.

These can be described by the general formula

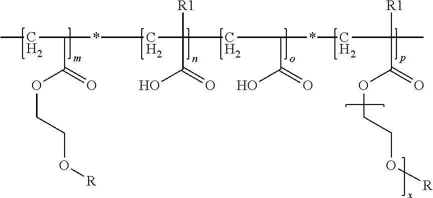

wherein m, n, o and p vary according to the molar mass of the polymer and are not intended to suggest that these are block copolymers. Rather, the structural units can be randomly distributed in the molecule. R1 is a methyl group, R a hydrocarbon residue with one to 22 C atoms, x is 0 to 50.

Particularly preferred compositions according to the invention contain as copolymer c4 copolymers of acrylic acid with methacrylic acid and ethoxylated acrylic acid esters and ethoxylated methacrylic acid esters having a molar mass of 100 to 500 kDa, preferably 150 to 400 kDa, more preferably 200 to 300 kDa and in particular 225 to 275 kDa.

Copolymers c4 are preferably used within defined quantity ranges. Compositions according to the invention are preferred here which contain, based on total weight of the ready-to-use compositions, 0.05 to 5 wt. %, preferably 0.1 to 4 wt. % and in particular 0.25 to 3 wt. % of copolymer(s) c4.

Particularly preferred copolymers c4 have 20 to 30 EO units (x=20, 21, 22, 23, 24, 25, 26, 27, 28, 29, 30) and have a stearyl residue or behenyl residue as the residue R.

A most particularly preferred copolymer c4 has 25 EO units, is esterified with behenyl alcohol, and is referred to under INCI nomenclature as Acrylates/Beheneth-25 Methacrylate Copolymer. Such a polymer is available, for example, under the trade name Aculyn® 28 (Rohm & Haas).

In addition to the polymer(s) c1 and/or copolymer(s) c2 and/or copolymer(s) c3 and/or copolymer(s) c4 or in its or their place, compositions according to the invention can also contain polymers c5 from the group of copolymers of acrylic acid esters with methacrylic acid.

Preferred acrylic acid esters are methyl acrylate and ethyl acrylate, the latter being particularly preferred.

Particularly preferred compositions according to the invention contain as copolymer c5 copolymers of acrylic acid esters with methacrylic acid having a molar mass of 100 to 500 kDa, preferably 150 to 400 kDa, more preferably 200 to 300 kDa and in particular 225 to 275 kDa.

Copolymers c5 are preferably used within defined quantity ranges. Compositions according to the invention are preferred here which, based on total weight of the ready-to-use compositions, contain 0.05 to 5 wt. %, preferably 0.1 to 4 wt. % and in particular 0.25 to 3 wt. % of copolymer(s) c5.

A most particularly preferred copolymer c5 derives from the polymerization of methacrylic acid with ethyl acrylate and is referred to under INCI nomenclature as Acrylates Copolymer. Such a polymer is available, for example, under the trade name Luviflex® Soft (BASF).

Most particularly preferred compositions according to the invention therefore contain a copolymer D of methacrylic acid and ethyl acrylate.

Copolymers D are preferably used which contain 10 to 80 mol %, preferably 20 to 70 mol %, particularly preferably 30 to 60 mol % and in particular 40 to 50 mol % of methacrylic acid and 20 to 90 mol %, preferably 30 to 80 mol %, particularly preferably 40 to 70 mol % and in particular 50 to 60 mol % of ethyl acrylate.

Compositions according to the invention are even more preferred wherein copolymer D has a molar mass of 100 to 800 kDa, preferably 200 to 700 kDa, more preferably 300 to 600 kDa and in particular 450 to 550 kDa.

Compositions according to the invention particularly preferably contain the copolymer sold by BASF AG under the name Luviflex® Soft (INCI name: Acrylates Copolymer).

Regardless of which copolymer(s) C or D is or are used, preferred compositions according to the invention are those wherein the total amount of copolymers C or D, based on total weight of the ready-to-use compositions, is 0.05 to 5 wt. %, preferably 0.1 to 4 wt. % and in particular 0.25 to 3 wt. %.

The present invention is subject to no restrictions regarding selection of copolymers B and C or D (D is a preferred polymer c5) that can additionally be used. Both a single polymer and multiple polymers from the individual classes described can be used in each case. Particularly preferred compositions contain in addition to copolymer A the further copolymers listed in the priority document under the references (1) to (217). Regardless of which of the 217 preferred polymer combinations is chosen, preferred compositions are those wherein the weight ratio of polymer(s) B to polymer(s) C is 10:1 to 1:10, preferably 8:1 to 1:8, more preferably 5:1 to 1:5 and in particular 4:1 to 1:4.

Regardless of the nature and weight ratio of the polymers to one another, compositions according to the invention are moreover preferred wherein the total polymer content (B+C+D) of the compositions is 1 to 15 wt. %, preferably 2.5 to 12.5 wt. %, more preferably 4 to 10 wt. % and in particular 5 to 8 wt. %.

In addition to the film-forming copolymers B and/or C and/or D or in their place, the compositions according to the invention can also contain film-forming polymers E from the group of acrylate polymers (i.e., polymers containing at least one monomer unit from the acrylic acid and/or methacrylic acid group and/or esters thereof). Preferred compositions according to the invention contain at least one copolymer E, formed from
at least one monomer e1 chosen from acrylic acid and/or methacrylic acid,
at least one monomer e2 chosen from acrylamide and/or methacrylamide, and
at least one monomer e3 chosen from N-substituted acrylamides and/or methacrylamides.

This copolymer E contains at least one monomer e1 chosen from acrylic acid and/or methacrylic acid, and at least one monomer e2 chosen from acrylamide and/or methacrylamide, and at least one monomer e3 chosen from N-substituted acrylamides and/or methacrylamides and can moreover have further structural units incorporated by addition of corresponding monomers during polymerization.

Particularly preferred copolymers A are copolymers of
acrylic acid and acrylamide and N-substituted acrylamides,
acrylic acid and methacrylamide and N-substituted acrylamides,
methacrylic acid and acrylamide and N-substituted acrylamides,
methacrylic acid and methacrylamide and N-substituted acrylamides and/or methacrylamides,
acrylic acid and acrylamide and N-substituted methacrylamides,
acrylic acid and methacrylamide and N-substituted methacrylamides,
methacrylic acid and acrylamide and N-substituted methacrylamides,
methacrylic acid and methacrylamide and N-substituted methacrylamides.

Particularly preferred compositions according to the invention have as copolymer E a copolymer E1 encompassing acrylic acid as the monomer e1. Acrylamide is a preferred monomer. Preferred compositions according to the invention thus contain as copolymer E a copolymer E1 encompassing acrylamide as the monomer e2.

The N-substitution at the N-substituted acrylamides can be carried out with simple alkyl groups (preferably methyl, ethyl, n-propyl, isopropyl); however, substituted alkyl groups bearing anionic functionalities are particularly preferred. Substituents containing sulfonate groups are most particularly preferred.

A particularly preferred composition according to the invention has as copolymer E a copolymer E1 encompassing acryloyldimethyltaurate as the monomer e3.

These copolymers E1 can be described by the general formula

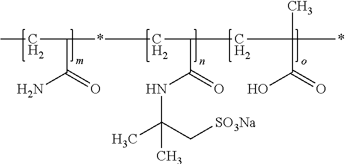

wherein m, n and o vary according to the molar mass of the polymer and are not intended to suggest that these are block copolymers. The structural units can rather be randomly distributed in the molecule.

Particularly preferred compositions according to the invention are those wherein copolymer E1 has a molar mass of 50 to 500 kDa, preferably 100 to 450 kDa, more preferably 150 to 400 kDa and in particular 200 to 300 kDa.

Copolymers E are preferably used within defined amounts. Preferred compositions according to the invention are those wherein, based on total weight of the ready-to-use compositions, the total amount of copolymers E is 0.05 to 5 wt. %, preferably 0.1 to 4 wt. % and in particular 0.25 to 3 wt. %.

Copolymers of acrylamide with methacrylic acid and acryloyldimethyltaurate are available, for example, under the trade name Acudyne® SCP (Rohm & Haas).

In addition to film-forming copolymers B and/or C and/or D and/or E or in their place, compositions according to the invention can further contain film-forming polymers F. Preferred compositions according to the invention contain at least one copolymer F chosen from
f1) copolymers of vinyl pyrrolidone with methacrylamidopropyl trimethylammonium chloride (MAPTAC), and/or
f2) copolymers of vinyl pyrrolidone with dimethylaminoethyl methacrylate, and/or
f3) copolymers of vinyl pyrrolidone with dimethylaminopropyl methacrylamide and alkyl dimethylpropyl methacrylamidoammonium salts.

Thus, preferred compositions according to the invention contain, for example, as polymer F copolymers of vinyl pyrrolidone with methacrylamidopropyl trimethylammonium chloride (MAPTAC) (b1).

These can be described by the general formula

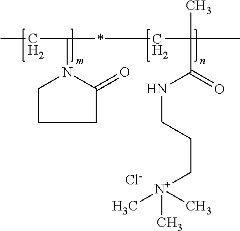

wherein m and n vary according to the molar mass of the polymer and are not intended to suggest that these are block copolymers. The structural units can rather be randomly distributed in the molecule.

Particularly preferred compositions according to the invention contain as the cationic polymer f1 copolymers of methacrylamidopropyl trimethylammonium chloride (MAPTAC) with vinyl pyrrolidone, containing 40 to 95 mol %, preferably 42.5 to 90 mol %, more preferably 45 to 85 mol % and in particular 50 to 80 mol % of vinyl pyrrolidone.

Particularly preferred compositions according to the invention are those wherein copolymers f1 have molar masses of 10 to 1000 kDa, preferably 25 to 900 kDa, more preferably 50 to 800 kDa and in particular 100 to 750 kDa.

Copolymers f1 are preferably used within defined amounts. Preferred compositions contain, based on total weight of the ready-to-use compositions, 0.05 to 5 wt. %, preferably 0.1 to 4 wt. % and in particular 0.25 to 3 wt % of copolymer(s) f1.

A most particularly preferred copolymer f1 is referred to under INCI nomenclature as Polyquaternium-28. Such a polymer is available, for example, under the trade name Gafquat® HS-100 (ISP).

In addition to polymer(s) f1 or in its or their place, compositions according to the invention can also contain polymers f2 from the group of copolymers of vinyl pyrrolidone with dimethylaminoethyl methacrylate.

These can be described by the general formula

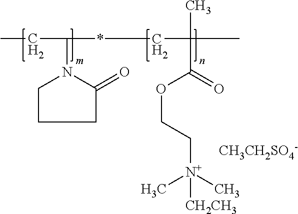

wherein m and n vary according to the molar mass of the polymer and are not intended to suggest that these are block copolymers. The structural units can rather be randomly distributed in the molecule.

Particularly preferred compositions according to the invention are those wherein as cationic polymer f2 they contain copolymers of vinyl pyrrolidone with dimethylaminoethyl methacrylate, containing 40 to 95 mol %, preferably 42.5 to 90 mol %, more preferably 45 to 85 mol % and in particular 50 to 80 mol % of vinyl pyrrolidone.

Particularly preferred compositions according to the invention are those wherein copolymers f2 have molar masses of 100 to 2500 kDa, preferably 250 to 2000 kDa, more preferably 500 to 1750 kDa and in particular 800 to 1500 kDa.

Copolymers f2 are preferably used within defined amounts. Preferred compositions contain, based on total weight of the ready-to-use compositions, 0.05 to 5 wt. %, preferably 0.1 to 4 wt. % and in particular 0.25 to 3 wt. % of copolymer(s) f2.

A most particularly preferred copolymer f2 is referred to under INCI nomenclature as Polyquaternium-11. Such a polymer is available, for example, under the trade name Gafquat® 755 N (ISP).

In addition to polymer(s) f1 and/or polymer(s) f2 or in its or their place, compositions according to the invention can also contain polymers f3 from the group of copolymers of vinyl pyrrolidone with dimethylaminopropyl methacrylamide and alkyl dimethylpropyl methacrylamidoammonium salts.

These can be described by the general formula

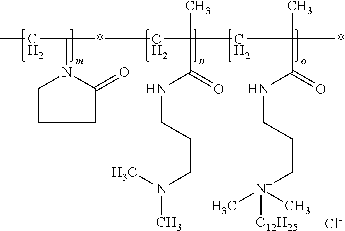

wherein m, n and o vary according to the molar mass of the polymer and are not intended to suggest that these are block copolymers. The structural units can rather be randomly distributed in the molecule.

Particularly preferred compositions according to the invention contain as cationic polymer f3 copolymers of vinyl pyrrolidone with dimethylaminopropyl methacrylamide and lauryl dimethylpropyl methacrylamidoammonium salts.

Particularly preferred compositions according to the invention further contain as cationic polymer f3 copolymers of vinyl pyrrolidone with dimethylaminopropyl methacrylamide and alkyl dimethylpropyl methacrylamidoammonium salts, containing 40 to 95 mol %, preferably 42.5 to 90 mol %, more preferably 45 to 85 mol % and in particular 50 to 80 mol % of vinyl pyrrolidone.

Most particularly preferred compositions according to the invention are those wherein copolymers f3 have molar masses of 10 to 1000 kDa, preferably 25 to 900 kDa, more preferably 50 to 800 kDa and in particular 100 to 750 kDa.

Copolymers f3 are preferably used within defined amounts. Preferred compositions contain, based on total weight of the ready-to-use compositions, 0.05 to 5 wt. %, preferably 0.1 to 4 wt. % and in particular 0.25 to 3 wt. % of copolymer(s) f3.

A most particularly preferred copolymer f3 is referred to under INCI nomenclature as Polyquaternium-55. Such a polymer is available, for example, under the trade name Styleze® W20 (ISP).

Regardless of the nature and weight ratio to one another of all polymers present in compositions according to the invention, preferred compositions are those in which the total polymer content of the compositions is 1 to 15 wt. %, preferably 2.5 to 12.5 wt. %, more preferably 4 to 10 wt. % and in particular 5 to 8 wt. %.

Agents according to the invention contain compound(s) having structural units of formula (I) in a cosmetic carrier. Creams, emulsions, gels or surfactant-containing foaming solutions, such as shampoos, foam aerosols or other preparations that are particularly suitable for use on hair are particularly suitable according to the invention as cosmetic carriers. It is also conceivable for the ingredients to be integrated into a formulation in powder or tablet form which is dissolved in water prior to application. The cosmetic carriers can in particular be aqueous or aqueous-alcoholic. An aqueous cosmetic carrier contains at least 50 wt. % of water.

Within the meaning of the present invention aqueous-alcoholic cosmetic carriers refer to aqueous solutions containing 3 to 70 wt. % of a C₁-C₆ alcohol, in particular methanol, ethanol or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol or 1,6-hexanediol. The agents can additionally contain further organic solvents such as methoxybutanol, benzyl alcohol, ethyl diglycol or 1,2-propylene glycol. All water-soluble organic solvents are preferred.

Agents according to the invention can contain further active ingredients and auxiliary substances. These are described below.

The agents preferably also contain at least one emulsifier or surfactant, wherein surface-active substances are referred to as surfactants or as emulsifiers depending on the area of application and are chosen from anionic, cationic, zwitterionic, ampholytic and non-ionic surfactants and emulsifiers.

Preferred hair treatment agents contain, based on total weight of the agent, 0.5 to 70 wt. %, preferably 1 to 60 wt. % and in particular 5 to 25 wt. % of anionic and/or non-ionic and/or cationic and/or amphoteric surfactant(s).

All anionic surface-active substances suitable for use on the human body are suitable as anionic surfactants and emulsifiers for compositions according to the invention. These have a water-solubilizing anionic group such as a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Preferred anionic surfactants and emulsifiers are acyl glutamates, acyl isethionates, acyl sarcosinates and acyl taurates, each having a linear or branched acyl residue with 6 to 22 carbon atoms and 0, 1, 2 or 3 double bonds, which in particularly preferred embodiments is chosen from an octanoyl, decanoyl, lauroyl, myristoyl, palmitoyl and stearoyl residue, esters of tartaric acid, citric acid or succinic acid or salts of these acids with alkylated glucose, in particular products with the INCI name Disodium Coco-Glucoside Citrate, Sodium Coco-Glucoside Tartrate and Disodium Coco-Glucoside Sulfosuccinate, alkyl polyglycol ether sulfates and ether carboxylic acids having 8 to 18 C atoms in the alkyl group and up to 12 ethoxy groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 ethoxy groups.

Surface-active compounds classified as zwitterionic surfactants and emulsifiers are those bearing at least one quaternary ammonium group and at least one —COO⁽⁻⁾ or —SO₃⁽⁻⁾ group in the molecule. Particularly suitable zwitterionic surfactants and emulsifiers are betaines such as N-alkyl-N,N-dimethylammonium glycinates, for example, cocoalkyl dimethylammonium glycinate, N-acyl aminopropyl-N,N-dimethylammonium glycinates, for example, cocoacyl aminopropyl dimethylammonium glycinate, and 2-alkyl-3-carboxymethyl-3-hydroxyethyl imidazolines, each having 8 to 18 C atoms in the alkyl or acyl group, as well as cocoacyl aminoethyl hydroxyethyl carboxymethyl glycinate. A preferred zwitterionic surfactant is the fatty acid amide derivative known under the INCI name Cocamidopropyl Betaine.

Ampholytic surfactants and emulsifiers are surface-active compounds which, in addition to a C₈-C₂₄ alkyl or acyl group, contain at least one free amino group and at least one —COOH or —SO₃H group and are capable of forming internal salts. Examples of suitable ampholytic surfactants are N-alkyl glycines, N-alkyl aminopropionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytic surfactants are N-cocoalkyl aminopropionate, cocoacylaminoethyl aminopropionate and C₁₂-C₁₈ acyl sarcosine.

Non-ionic surfactants and emulsifiers contain as a hydrophilic group a polyol group, a polyalkylene glycol ether group or a combination of polyol and polyglycol ether group, for example.

Alkyl polyglycosides, optionally mixed with fatty alcohols, alkoxylated polydialkyl siloxanes, alkylene oxide addition products with saturated linear fatty alcohols and fatty acids each containing 2 to 30 mol of ethylene oxide per mol of fatty alcohol or fatty acid are preferred non-ionic surface-active substances.

Also suitable for use according to the invention are cationic surfactants of the quaternary ammonium compound, esterquat and amidoamine type. Preferred quaternary ammonium compounds are ammonium halides, in particular chlorides and bromides such as alkyl trimethylammonium chlorides, dialkyl dimethylammonium chlorides and trialkyl methylammonium chlorides. The long alkyl chains of these surfactants preferably have 10 to 18 carbon atoms, such as in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are imidazolium compounds known under the INCI names Quaternium-27 and Quaternium-83.

Most particularly preferred agents according to the invention additional contain fatty alcohol(s) and/or fatty alcohol alkoxylate(s), preferably C₁₂₋₂₂ fatty alcohol(s) and/or C₁₂₋₂₂ fatty alcohol ethoxylate(s) having 10 to 30 EO units, particularly preferably C₁₆₋₁₈ fatty alcohol(s) and/or C₁₆₋₁₈ fatty alcohol ethoxylate(s) having 12 to 20 EO units, preferably in amounts from 5 to 20 wt. %, more preferably 7.5 to 17.5 wt. % and in particular 10 to 15 wt. %, based on total weight of the agent.

In summary, preferred hair treatment agents according to the invention contain, based on total weight of the agent, 0.1 to 20 wt. %, preferably 0.25 to 17.5 wt. % and in particular 5 to 15 wt. % of anionic surfactant(s), particularly preferably fatty alcohol ether sulfates of the formula

H₃C—(CH₂)ₙ—(OCH₂CH₂)ₖ—OSO₃⁻M⁺

wherein n is a value from 5 to 21, preferably 7 to 19, more preferably 9 to 17, and in particular 11 to 13; k is a value of 1, 2, 3, 4, 5, 6, 7, 8, 9 or 10, preferably 1, 2 or 3 and in particular 2; and M is a cation from the group Na⁺, K⁺NH₄⁺, ½Mg²⁺, ½Zn²⁺, preferably Na⁺.

Preferred hair treatment agents according to the invention can additionally contain amphoteric surfactant(s), preferably from the groups of N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids each having approximately 8 to 24 C atoms in the alkyl group, alkyl aminoacetic acids each having approximately 8 to 24 C atoms in the alkyl group, N-cocoalkyl aminopropionate, cocoacyl aminoethyl aminopropionate, C₁₂-C₁₈ acyl sarcosine, N-alkyl-N,N-dimethylammonium glycinates, for example, cocoalkyl dimethylammonium glycinate, N-acyl aminopropyl-N,N-dimethylammonium glycinates, for example, cocoacyl aminopropyl dimethylammonium glycinate, 2-alkyl-3-carboxymethyl-3-hydroxyethyl imidazolines each having 8 to 18 C atoms in the alkyl or acyl group, cocoacyl aminoethyl hydroxyethyl carboxymethyl glycinate, compounds known under the INCI name Cocamidopropyl Betaine, compounds known under the INCI name Disodium Cocoamphodiacetate. Preferred agents contain the amphoteric surfactant(s) in amounts from 1 to 15 wt. %, preferably from 2.5 to 12 wt. % and in particular from 5 to 10 wt. %, based on total weight of the agent.

Preferred hair treatment agents according to the invention contain as amphoteric surfactants betaines of the formula (B-I)

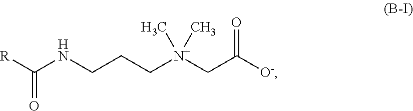

wherein R is a straight-chain or branched, saturated or mono- or polyunsaturated alkyl or alkenyl residue having 8 to 24 carbon atoms.

Under INCI nomenclature these surfactants are known as Amidopropylbetaine, wherein representatives derived from coconut fatty acids are preferred and are known as Cocoamidopropylbetaine. Surfactants of formula (B-I) which are a mixture of the following representatives are particularly preferably used according to the invention: H₃C—(CH₂)₇—C(O)—NH—(CH₂)₃N⁺(CH₃)₂CH₂COO⁻, H₃C—(CH₂)₉—C(O)—NH—(CH₂)₃N⁺(CH₃)₂CH₂COO⁻, H₃C—(CH₂)₁₁—C(O)—NH—(CH₂)₃N⁺(CH₃)₂CH₂COO⁻, H₃C—(CH₂)₁₃—C(O)—NH—(CH₂)₃N⁺(CH₃)₂CH₂COO⁻, H₃C—(CH₂)₁₅—C(O)—NH—(CH₂)₃N⁺(CH₃)₂CH₂COO⁻, H₃C—(CH₂)₇CH═CH—(CH₂)₇—C(O)—NH—(CH₂)₃N⁺(CH₃)₂CH₂COO⁻.

Surfactants of formula (I) are particularly preferably used within narrower amounts. Preferred agents according to the invention here contain, relative to their weight, 0.25 to 8 wt. %, more preferably 0.5 to 7 wt. %, more preferably 0.75 to 6.5 wt. % and in particular 1 to 5.5 wt. % of surfactant(s) of formula (B-I).

In addition to the amphoteric surfactants of formula (I) or in their place, hair treatment agents according to the invention can contain with particular preference as amphoteric surfactants betaines of formula (B-II)

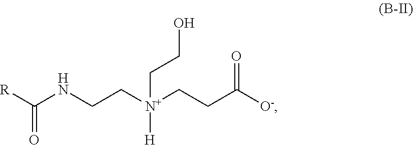

wherein R is a straight-chain or branched, saturated or mono- or polyunsaturated alkyl or alkenyl residue having 8 to 24 carbon atoms.

Under INCI nomenclature these surfactants are known as Amphoacetate, wherein representatives derived from coconut fatty acids are preferred and are known as Cocoamphoactetate.

For production reasons, surfactants of this type always also contain betaines of formula (B-IIa)

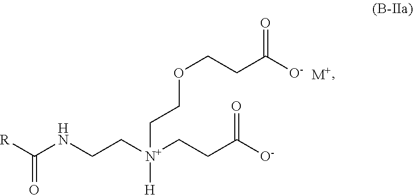

wherein R is a straight-chain or branched, saturated or mono- or polyunsaturated alkyl or alkenyl residue having 8 to 24 carbon atoms and M is a cation.

Under INCI nomenclature these surfactants are known as Amphodiacetate, wherein representatives derived from coconut fatty acids are preferred and are known as Cocoamphodiacetate.

Surfactants of formula (B-II) which are a mixture of the following representatives are particularly preferably used according to the invention:
H₃C—(CH₂)₇—C(O)—NH—(CH₂)₂NH⁺(CH₂CH₂OH)CH₂CH₂COO⁻
H₃C—(CH₂)₉—C(O)—NH—(CH₂)₂NH⁺(CH₂CH₂OH)CH₂CH₂COO⁻
H₃C—(CH₂)₁₁—C(O)—NH—(CH₂)₂NH⁺(CH₂CH₂OH)CH₂CH₂COO⁻
H₃C—(CH₂)₁₃—C(O)—NH—(CH₂)₂NH⁺(CH₂CH₂OH)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂NH⁺(CH₂CH₂OH)CH₂CH₂COO⁻

Surfactants of formula (B-II) are particularly preferably used within narrower quantity ranges. Agents according to the invention are preferred here which, relative to their weight, contain 0.25 to 8 wt. %, more preferably 0.5 to 7 wt. %, more preferably 0.75 to 6.5 wt. % and in particular 1 to 5.5 wt. % of surfactant(s) of formula (B-II).

In summary, hair treatment agents according to the invention are preferred wherein R in formulae (B-I) and (B-II) is chosen from H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇— or mixtures thereof.

Particularly preferred non-ionic surfactants are alkyl polyglycosides. Preferred hair treatment agents thus contain, as non-ionic surfactants, alkyl polyglycosides of the general formula RO—(Z)ₓ, wherein R is alkyl, Z is sugar and x is the number of sugar units.

Alkyl polyglycosides corresponding to the general formula RO—(Z)ₓ wherein R is alkyl, Z is sugar and x is the number of sugar units, are preferably used according to the invention. Such alkyl polyglycosides are particularly preferred wherein R consists substantially of C₈ and C₁₀ alkyl groups, substantially of C₁₂ and C₁₄ alkyl groups, substantially of C₈ to C₁₆ alkyl groups, substantially of C₁₂ to C₁₆ alkyl groups or substantially of C₁₆ to C₁₈ alkyl groups.

Any mono- or oligosaccharides can be used as the sugar structural unit Z. Sugars having 5 or 6 carbon atoms and the corresponding oligosaccharides are conventionally used. Such sugars include glucose, fructose, galactose, arabinose, ribose, xylose, lyxose, allose, altrose, mannose, gulose, idose, talose and sucrose. Preferred sugar structural units are glucose, fructose, galactose, arabinose and sucrose; glucose is particularly preferred.

Alkyl polyglycosides for use according to the invention contain on average 1.1 to 5 sugar units. Alkyl polyglycosides having x values from 1.1 to 2.0 are preferred. Alkyl glycosides wherein x is 1.1 to 1.8 are most particularly preferred.

In addition to the specified surfactants, agents according to the invention can also contain anionic, cationic and optionally amphoteric or zwitterionic or non-ionic surfactants.

Particularly preferred hair treatment agents as cationic care substance contain, based on their weight, 0.05 to 7.5 wt. %, preferably 0.1 to 5 wt. %, more preferably 0.2 to 3.5 wt. % and in particular 0.25 to 2.5 wt. % of cationic surfactant(s) from the group of quaternary ammonium compounds and/or esterquats and/or amidoamines, wherein preferred cationic surfactant(s) is/are chosen from
alkyl trimethylammonium chlorides preferably having 10 to 18 carbon atoms in the alkyl residue,
dialkyl dimethylammonium chlorides preferably having 10 to 18 carbon atoms in the alkyl residue,
trialkyl methylammonium chlorides preferably having 10 to 18 carbon atoms in the alkyl residue,
cetyl trimethylammonium chloride,
stearyl trimethylammonium chloride,
distearyl dimethylammonium chloride,
lauryl dimethylammonium chloride,
lauryl dimethyl benzylammonium chloride,
tricetyl methylammonium chloride,
Quaternium-27,
Quaternium-83,
N-methyl-N-(2-hydroxyethyl)-N,N-(ditallow acyloxyethyl)ammonium methosulfate,
N-methyl-N-(2-hydroxyethyl)-N,N-(distearoyloxyethyl)ammonium methosulfate,
N,N-dimethyl-N,N-distearoyloxyethyl ammonium chloride, and
N,N-di-(2-hydroxyethyl)-N,N-(fatty acid ester ethyl)ammonium chloride.

As a further optional constituent, agents according to the invention can contain 0.01 to 10 wt. % of at least one polymer from the group of cationic and/or amphoteric polymers. These are described in the priority document on pages 227 to 231.

In summary, preferred hair treatment agents according to the invention contain, based on their weight, 0.05 to 7.5 wt. %, preferably 0.1 to 5 wt. %, particularly preferably 0.2 to 3.5 wt. % and in particular 0.25 to 2.5 wt. % of cationic polymer(s), wherein preferred cationic polymer(s) is/are chosen from poly(methacryloyloxyethyl trimethylammonium chloride) (INCI: Polyquaternium-37) and/or quaternized cellulose derivatives (INCI: Polyquaternium 10) and/or cationic alkyl polyglycosides and/or cationized honey and/or cationic guar derivatives and/or polymeric dimethyl diallyl ammonium salts and copolymers thereof with esters and amides of acrylic acid and methacrylic acid and/or copolymers of vinyl pyrrolidone with quaternized derivatives of dialkyl aminoalkyl acrylate and methacrylate and/or vinyl pyrrolidone-vinyl imidazolium methochloride copolymers and/or quaternized polyvinyl alcohol and/or Polyquaternium 2 and/or Polyquaternium 7 and/or Polyquaternium 17 and/or Polyquaternium 18 and/or Polyquaternium 24 and/or Polyquaternium 27.

In addition to the cationic polymers or in their place, agents according to the invention can contain amphoteric polymers. In the context of the present invention, amphoteric polymers which can preferably be used are substantially composed of
i) monomers having quaternary ammonium groups of general formula (Z-I),

R¹—CH═CR²—CO—Z—(CₙH₂ₙ)—N⁽⁺⁾R³R⁴R⁵A⁽⁻⁾ (Z-I)
wherein R¹ and R² independently of each other are hydrogen or a methyl group and R³, R⁴ and R⁵ independently of one another are alkyl groups having 1 to 4 carbon atoms, Z is an NH group or an oxygen atom, n is a whole number from 2 to 5, and A⁽⁺⁾ is the anion of an organic or inorganic acid, and
ii) monomeric carboxylic acids of general formula (Z-II),

R⁶—CH═CR⁷—COOH (Z-II)
wherein R⁶ and R⁷ are independently of each other hydrogen or methyl groups.

Suitable starting monomers include dimethyl aminoethyl acrylamide, dimethyl aminoethyl methacrylamide, dimethyl aminopropyl acrylamide, dimethyl aminopropyl methacrylamide and diethyl aminoethyl acrylamide if Z is an NH group, or dimethyl aminoethyl acrylate, dimethyl aminoethyl methacrylate and diethyl aminoethyl acrylate if Z is oxygen.

Monomers containing a tertiary amino group are then quaternized in a known manner, wherein methyl chloride, dimethyl sulfate or diethyl sulfate are particularly suitable as alkylating reagents. The quaternization reaction can take place in aqueous solution or in solvent.

Such monomers of formula (Z-I) which are derivatives of acrylamide or methacrylamide are advantageously used. Such monomers having as counterions halide, methoxysulfate or ethoxysulfate ions are furthermore preferred. Such monomers of the formula (Z-I) wherein R³, R⁴ and R⁵ are methyl groups are likewise preferred.

Acrylamidopropyl trimethylammonium chloride is a most particularly preferred monomer of formula (Z-I).

Acrylic acid, methacrylic acid, crotonic acid and 2-methylcrotonic acid are suitable as monomeric carboxylic acids of formula (Z-II). Acrylic or methacrylic acid is preferably used, in particular acrylic acid.

Zwitterionic polymers which can be used according to the invention are produced from monomers of formulae (Z-I) and (Z-II) by polymerization methods known per se. Polymerization can take place in either aqueous or aqueous-alcoholic solution. Alcohols having 1 to 4 carbon atoms, preferably isopropanol, which simultaneously serve as polymerization regulators are used as alcohols. Other components can, however, also be added to the monomer solution as regulators, for example, formic acid or mercaptans such as thioethanol and thioglycolic acid. The polymerization is initiated with the aid of radical-forming substances. Redox systems and/or thermally decomposing radical formers of the azo compound type, such as azoisobutyric acid nitrile, azo bis-(cyanopentanoic acid) or azo bis(amidinopropane)dihydrochloride, can be used. Combinations of hydrogen peroxide, potassium or ammonium peroxodisulfate and tertiary butyl hydroperoxide with sodium sulfite, sodium dithionite or hydroxylamine hydrochloride as the reduction component are suitable as redox systems.

Polymerization can be performed isothermally or under adiabatic conditions, wherein depending on the concentration ratios, the temperature range for the reaction course can vary from 20 to 200° C. due to the liberating heat of polymerization, and the reaction optionally has to be performed under excess pressure that is established. The reaction temperature is preferably from 20 to 100° C.

The pH can vary within a broad range during copolymerization. Polymerization is advantageously performed at low pH values, but pH values above the neutral point are also possible. After polymerization, the pH is adjusted to a value from 5 to 10, preferably from 6 to 8, with an aqueous base such as sodium hydroxide solution, potassium hydroxide solution or ammonia. Further details about the polymerization method can be taken from the Examples.

Such polymers have proven particularly effective wherein monomers of formula (Z-I) were present in excess in comparison to monomers of formula (Z-II). It is therefore preferable according to the invention to use polymers consisting of monomers of formula (Z-I) and monomers of formula (Z-II) in a molar ratio of 60:40 to 95:5, in particular 75:25 to 95:5.

Preferred agents according to the invention are those wherein the amphoteric polymer(s) encompass monomers A) and B), wherein A) and B) are chosen from
i) monomers having quaternary ammonium groups of general formula (Z-I),

R¹—CH═CR²—CO—Z—(CₙH₂ₙ)—N⁽⁺⁾R³R⁴R⁵A⁽⁻⁾ (Z-I)
wherein R¹ and R² independently of each other are hydrogen or a methyl group and R³, R⁴ and R⁵ independently of one another are alkyl groups having 1 to 4 carbon atoms, Z is an NH group or an oxygen atom, n is a whole number from 2 to 5 and A⁽⁻⁾ is the anion of an organic or inorganic acid, and
ii) monomeric carboxylic acids of general formula (Z-II),

R⁶—CH═CR⁷—COOH (Z-II)
wherein R⁶ and R⁷ are independently of each other hydrogen or methyl groups.

Amphoteric polymers used in agents according to the invention contain to particular advantage monomers from the group of acrylamides and/or methacrylamides having alkyl ammonium groups. Acrylic acid and/or methacrylic acid and/or crotonic acid and/or 2-methylcrotonic acid have proven effective as monomers having anionic groups additionally present in the polymers.

In summary, preferred agents according to the invention are those wherein the amphoteric polymer(s) are copolymers of at least one of the monomers trimethylammonium ethyl acrylamide and/or trimethylammonium ethyl methacrylamide and/or trimethylammonium propyl acrylamide and/or trimethylammonium propyl methacrylamide and/or trimethylammonium ethyl acrylamide and/or trimethylammonium ethyl acrylate and/or trimethylammonium ethyl methacrylate and/or trimethylammonium ethyl acrylate and/or ethyl dimethylammonium ethyl acrylamide and/or ethyl dimethylammonium ethyl methacrylamide and/or ethyl dimethylammonium propyl acrylamide and/or ethyl dimethylammonium propyl methacrylamide and/or ethyl dimethylammonium ethyl acrylamide and/or ethyl dimethylammonium ethyl acrylate and/or ethyl dimethylammonium ethyl methacrylate and/or ethyl dimethylammonium ethyl acrylate with at least one of the monomers acrylic acid and/or methacrylic acid and/or crotonic acid and/or 2-methylcrotonic acid.

Amphoteric polymers that are particularly preferred according to the invention are described in the priority document on pages 235 and 236.

As a further ingredient, agents according to the invention can contain one or more amino acids to particular advantage. Amino acids that can be particularly preferably used derive from the group comprising glycine, alanine, valine, leucine, isoleucine, phenylalanine, tyrosine, tryptophane, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophane, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used. Preferred agents contain one or more amino acids in narrower quantity ranges. Preferred hair treatment agents contain as a care substance, based on their weight, 0.01 to 5 wt. %, preferably 0.02 to 2.5 wt. %, particularly preferably 0.05 to 1.5 wt. %, more preferably 0.075 to 1 wt. % and in particular 0.1 to 0.25 wt. % of amino acid(s), preferably from the group comprising glycine and/or alanine, and/or valine and/or lysine and/or leucine and/or threonine.

A further preferred group of ingredients of agents according to the invention are vitamins, provitamins or vitamin precursors. In summary, preferred hair treatment agents according to the invention additionally contain as a care substance, based on their weight, 0.1 to 5 wt. %, preferably 0.2 to 4 wt. %, particularly preferably 0.25 to 3.5 wt. %, more preferably 0.5 to 3 wt. % and in particular 0.5 to 2.5 wt. % of vitamins and/or provitamins and/or vitamin precursors, preferably assigned to groups A, B, C, E, F and H, wherein preferred agents contain panthenol ((±)-2,4-dihydroxy-N-(3-hydroxypropyl)-3,3-dimethyl butyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂.

It has been found that use of certain quinones strengthens an anti-dandruff and anti-hair loss effect and improves combability and shine. As a further constituent, agents according to the invention can therefore contain 0.0001 to 5 wt. % of at least one bioquinone of formula (Ubi)

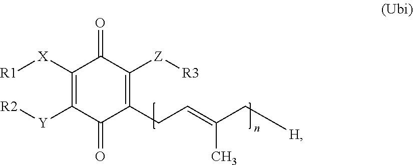

wherein
X, Y, Z independently of one another are —O— or —NH— or NR⁴— or a chemical bond;
R¹, R², R³ independently of each other are a hydrogen atom or an optionally substituted aryl group or an optionally substituted (C₁-C₆) alkyl group or a hydroxyalkyl group or a polyhydroxyalkyl group or an optionally substituted (C₁-C₆) alkylene group, or a (C₁-C₆) acyl residue, wherein preferred residues are chosen independently of one another from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃;
R⁴ is —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃; and
n is a value from 1 to 20, preferably 2 to 15, and in particular 5, 6, 7, 8, 9, 10.

Preferred compounds of formula (Ubi) according to the invention include the compounds disclosed in the priority document under the names (Ubi-1) to (Ubi-7).

Particularly preferred hair treatment agents according to the invention contain as a care substance they, based on their weight, 0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein preferred agents contain an ubiquinone of the formula (Ubi) (see above), in which n is a value of 6, 7, 8, 9 or 10, particularly preferably 10 (coenzyme Q10).

As an alternative to or in addition to the particularly preferred ubiquinones, agents according to the invention can also contain plastoquinones. Preferred agents contain 0.0002 to 4 wt. %, preferably 0.0005 to 3 wt. %, particularly preferably 0.001 to 2 wt. %, more preferably 0.0015 to 1 and in particular 0.002 to 0.5 wt. % of at least one plastoquinone of formula (Ubi-Ib)

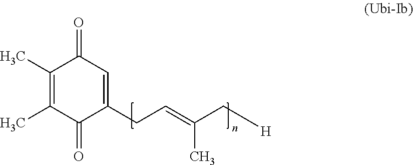

wherein n is a value from 1 to 20, preferably 2 to 15 and in particular 5, 6, 7, 8, 9, 10, wherein agents particularly preferably contain plastoquinone PQ-9 of the formula

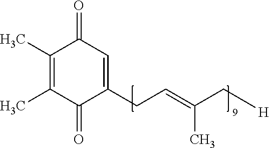

To improve the elasticity and strength of the internal structure of hair treated with agents according to the invention, the agents can contain purine and/or purine derivatives. In particular, the combination of purine and/or purine derivatives with ubiquinones and/or plastoquinones means that hair treated with corresponding agents exhibits inter alia higher measured values in differential thermal analysis and improved wet and dry combability.

As a further ingredient, agents according to the invention can therefore contain purine and/or purine derivative(s). Preferred agents contain purine and/or purine derivatives in narrower quantities. Preferred cosmetic agents according to the invention contain, relative to their weight, 0.001 to 2.5 wt. %, preferably 0.0025 to 1 wt. %, more preferably 0.005 to 0.5 wt. % and in particular 0.01 to 0.1 wt. % of purine(s) and/or purine derivative(s).

Some representatives of purine, purines and purine derivatives are particularly preferred according to the invention. Preferred hair treatment agents contain as a care substance, relative to their weight, 0.001 to 2.5 wt. %, preferably 0.0025 to 1 wt. %, more preferably 0.005 to 0.5 wt. % and in particular 0.01 to 0.1 wt. % of purine(s) and/or purine derivative(s), wherein preferred agents contain purine(s) and/or purine derivative(s) of formula (Pur-I)

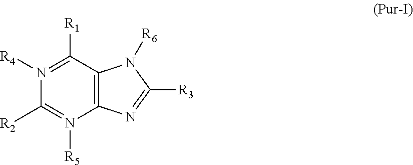

wherein R¹, R² and R³ are independently chosen from —H, —OH, NH₂, —SH; and R⁴, R⁵ and R⁶ are independently chosen from —H, —CH₃ and —CH₂—CH₃, with the following compounds being preferred: purine (R¹═R²═R³═R⁴═R⁵═R⁶═H), adenine (R¹═NH₂, R²═R³═R⁴═R⁵═R⁶═H), guanine (R¹═OH, R²═NH₂, R³═R⁴═R⁵═R⁶═H), uric acid (R¹═R²═R³═OH, R⁴═R⁵═R⁶═H), hypoxanthine (R¹═OH, R²═R³═R⁴═R⁵═R⁶═H), 6-purinethiol (R¹═SH, R²═R³═R⁴═R⁵═R⁶═H), 6-thioguanine (R¹═SH, R²═NH₂, R³═R⁴═R⁵═R⁶═H), xanthine (R¹═R²═OH, R³═R⁴═R⁵═R⁶═H), caffeine (R¹═R²═OH, R³═H, R⁴═R⁵═R⁶═CH₃), theobromine (R¹═R²═OH, R³═R⁴═H, R⁵═R⁶═CH₃), theophylline (R¹═R²═OH, R³═H, R⁴═CH₃, R⁵═CH₃, R⁶═H).

Depending on the desired application of the agents, the nature and amount of the purine derivative can vary. In hair cosmetics formulations, caffeine, which can be used in shampoos, for example, preferably in amounts from 0.005 to 0.25 wt. %, more preferably from 0.01 to 0.1 wt. % and in particular from 0.01 to 0.05 wt. % (relative in each case to the shampoo), has proven effective in particular.

It is furthermore advantageous to use purine or purine derivatives and bioquinones in a defined ratio to one another. Preferred agents are those wherein the weight ratio of ingredients a) and b) is 10:1 to 1:100, preferably 5:1 to 1:50, particularly preferably 2:1 to 1:20 and in particular 1:1 to 1:10.

As has already been mentioned, caffeine is a particularly preferred purine derivative, and coenzyme Q10 is a particularly preferred bioquinone. Particularly preferred agents according to the invention therefore contain, relative to their weight, 0.001 to 2.5 wt. %, preferably 0.0025 to 1 wt. %, particularly preferably 0.005 to 0.5 wt.-% and in particular 0.01 to 0.1 wt. % of caffeine and 0.0002 to 4 wt. %, preferably 0.0005 to 3 wt. %, particularly preferably 0.001 to 2 wt. %, more preferably 0.0015 to 1 and in particular 0.002 to 0.5 wt. % of coenzyme Q10.

As a further ingredient, agents according to the invention can contain at least one carbohydrate from the group of monosaccharides, disaccharides and/or oligosaccharides. Preferred hair treatment agents contain as a care substance, relative to their weight, 0.01 to 5 wt. %, preferably 0.05 to 4.5 wt. %, particularly preferably 0.1 to 4 wt. %, more preferably 0.5 to 3.5 wt. % and in particular 0.75 to 2.5 wt. % of carbohydrate(s) chosen from monosaccharides, disaccharides and/or oligosaccharides, wherein preferred carbohydrates are chosen from monosaccharides, in particular D-ribose and/or D-xylose and/or L-arabinose and/or D-glucose and/or D-mannose and/or D-galactose and/or D-fructose and/or sorbose and/or L-fucose and/or L-rhamnose, disaccharides, in particular sucrose and/or maltose and/or lactose and/or trehalose and/or cellobiose and/or gentiobiose and/or isomaltose.

Particularly preferred agents according to the invention contain, relative to their weight,
0.005 to 0.015 wt. % caffeine and 0.75 to 1.5 wt. % glucose monohydrate,
0.005 to 0.015 wt. % caffeine and 0.75 to 1.5 wt. % sucrose,
0.005 to 0.015 wt. % caffeine and 0.75 to 1.5 wt. % fructose.

As previously mentioned, preferred agents according to the invention contain (an) amino acid(s).

Amino acids that can particularly preferably be used according to the invention include glycine, alanine, valine, leucine, isoleucine, phenylalanine, tyrosine, tryptophane, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophane, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention contain one or more amino acids in narrower quantity ranges. Preferred cosmetic agents additionally contain 0.05 to 5 wt. %, preferably 0.1 to 2.5 wt. %, more preferably 0.15 to 1 wt. % and in particular 0.2 to 0.5 wt. % of amino acid(s), preferably (an) amino acid(s) from the group comprising glycine and/or alanine, and/or valine and/or lysine and/or leucine and/or threonine.

Particularly preferred agents according to the invention contain, relative to their weight,
0.005 to 0.015 wt. % caffeine, 0.75 to 1.5 wt. % glucose monohydrate and 0.1 to 0.25 wt. % glycine,
0.005 to 0.015 wt. % caffeine, 0.75 to 1.5 wt. % sucrose and 0.1 to 0.25 wt. % glycine,
0.005 to 0.015 wt. % caffeine, 0.75 to 1.5 wt. % fructose and 0.1 to 0.25 wt. % glycine,
0.005 to 0.015 wt. % caffeine, 0.75 to 1.5 wt. % glucose monohydrate and 0.1 to 0.25 wt. % alanine,
0.005 to 0.015 wt. % caffeine, 0.75 to 1.5 wt. % sucrose and 0.1 to 0.25 wt. % alanine,
0.005 to 0.015 wt. % caffeine, 0.75 to 1.5 wt. % fructose and 0.1 to 0.25 wt. % alanine,
0.005 to 0.015 wt. % caffeine, 0.75 to 1.5 wt. % glucose monohydrate and 0.1 to 0.25 wt. % valine,
0.005 to 0.015 wt. % caffeine, 0.75 to 1.5 wt. % sucrose and 0.1 to 0.25 wt. % valine,
0.005 to 0.015 wt. % caffeine, 0.75 to 1.5 wt. % fructose and 0.1 to 0.25 wt. % valine.

Preferred agents according to the invention contain as a care substance, relative to their weight, 0.01 to 15 wt. %, preferably 0.025 to 12.5 wt. %, more preferably 0.05 to 10 wt. %, even more preferably 0.1 to 7.5 wt. % and in particular 0.5 to 5 wt. % of at least one 2-furanone derivative of formula (Fur-I) and/or of formula (Fur-II), which are described in the priority document on pages 245 to 250.

In a most particularly preferred embodiment, dihydro-3-hydroxy-4,4-dimethyl-2(3H)-furanone is used as the compound corresponding to the formula (Fur-I).

A further care substance having activating properties that can preferably be used is taurine. Preferred hair treatment agents contain as a care substance, relative to their weight, 0.01 to 15 wt. %, preferably 0.025 to 12.5 wt. %, particularly preferably 0.05 to 10 wt. %, more preferably 0.1 to 7.5 wt. % and in particular 0.5 to 5 wt. % of taurine (2-aminoethane sulfonic acid).

Also preferred is the additional use of bisabolol and/or bisabolol oxides in agents according to the invention. Preferred hair treatment agents additionally contain 0.001 to 5 wt. %, preferably 0.01 to 4 wt. %, particularly preferably 0.02 to 2.5 wt. % and in particular 0.1 to 1.5 wt. % of bisabolol and/or oxides of bisabolol, preferably (−)-alpha-bisabolol.

Agents according to the invention can additionally contain further substances which prevent, alleviate or cure hair loss. A content of active ingredients which stabilize the hair root is particularly advantageous. In summary, preferred cosmetic agents according to the invention additionally contain, relative to their weight, 0.001 to 5 wt. % of hair root-stabilizing substances, in particular minoxidil and/or finasteride and/or ketoconazole.

Through additional anti-dandruff active ingredients (e.g., climbazole, piroctone olamine or zinc pyrithione), the amount of dandruff-causing fungal yeast is selectively reduced, bacterial flora return to their normal percentage composition and scaling is reduced to the physiological level. Laboratory tests have shown, however, that the various species representatives of Pityrosporum ovale respond with varying success to the anti-dandruff active ingredients. To combat all causes of dandruff with maximum effectiveness, a combination of anti-dandruff active ingredients is therefore most successful.

In summary, hair treatment agents according to the invention are preferred which additionally contain, relative to their weight, 0.001 to 5 wt. % of anti-dandruff active ingredients, in particular piroctone olamine (1-hydroxy-4-methyl-6-(2,4,4-trimethylpentyl)pyridin-2(1H)-one, compound with 2-aminoethanol, 1:1) and/or zinc pyrithione and/or selenium sulfide and/or climbazole and/or salicylic acid or fumaric acid.

Agents according to the invention can furthermore contain all active ingredients, additives and auxiliary substances known for such preparations. Often the agents contain at least one surfactant, with both anionic and zwitterionic, ampholytic, non-ionic and cationic surfactants being suitable in principle. It has proved advantageous in many cases, however, to choose surfactants from anionic, zwitterionic or non-ionic surfactants. These surfactants have been previously described in detail.

In a further preferred embodiment, the agents can contain emulsifiers (F). The agents contain emulsifiers preferably in amounts from 0.1 to 25 wt. %, in particular 0.5 to 15 wt. %, based on total agent.

Compositions according to the invention can preferably contain at least one non-ionogenic emulsifier having an HLB value of 8 to 18. Non-ionogenic emulsifiers having an HLB value of 10 to 15 can be particularly preferred according to the invention.

It has further proved advantageous if, in addition to polymer(s) from the group of cationic and/or amphoteric polymers, further polymers (G) are present in agents according to the invention. In a preferred embodiment further polymers are therefore added to the agents according to the invention, both anionic and non-ionic polymers having proven effective.

Anionic polymers (G2) are anionic polymers having carboxylate and/or sulfonate groups. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid.

Anionic polymers containing as the sole monomer or as a co-monomer 2-acrylamido-2-methylpropane sulfonic acid, wherein some or all of the sulfonic acid group can be present as the sodium, potassium, ammonium, mono- or triethanolammonium salt, have proved to be most particularly effective.

The homopolymer of 2-acrylamido-2-methylpropane sulfonic acid, commercially available, for example, under the name Rheothik® 11-80, is particularly preferred.

Within this embodiment it can be preferred to use copolymers consisting of at least one anionic monomer and at least one non-ionogenic monomer. Reference is made to the aforementioned substances with regard to the anionic monomers. Preferred non-ionogenic monomers are acrylamide, methacrylamide, acrylic acid ester, methacrylic acid ester, vinyl pyrrolidone, vinyl ether and vinyl ester.

Preferred anionic copolymers are acrylic acid-acrylamide copolymers as well as in particular polyacrylamide copolymers with monomers containing sulfonic acid groups. A particularly preferred anionic copolymer consists of 70 to 55 mol % of acrylamide and 30 to 45 mol % of 2-acrylamido-2-methylpropane sulfonic acid, wherein some or all of the sulfonic acid group is present as the sodium, potassium, ammonium, mono- or triethanolammonium salt. This copolymer can also be crosslinked, wherein polyolefinically unsaturated compounds such as tetraallyl oxyethane, allyl sucrose, allyl pentaerythritol and methylene bisacrylamide are preferably used as crosslinking agents. Such a polymer is present in the commercial product Sepigel®305 from SEPPIC. Use of this compound, which in addition to the polymer component contains a mixture of hydrocarbons (C₁₃-C₁₄ isoparaffin) and a non-ionogenic emulsifier (Laureth-7), has proved to be particularly advantageous within the context of the teaching according to the invention.

Sodium acryloyldimethyltaurate copolymers sold under the name Simulgel® 600 as a compound with isohexadecane and Polysorbate-80 have also proved to be particularly effective according to the invention.

Likewise preferred anionic homopolymers are uncrosslinked and crosslinked polyacrylic acids. Allyl ethers of pentaerythritol, sucrose and propylene can be preferred crosslinking agents. Such compounds are commercially available, for example, under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

In a further embodiment, agents according to the invention can contain non-ionogenic polymers (G4).

Suitable non-ionogenic polymers include:
vinyl pyrrolidone/vinyl ester copolymers, such as those sold under the trademark Luviskol® (BASF); Luviskol® VA 64 and Luviskol® VA 73, both of which are vinyl pyrrolidone/vinyl acetate copolymers, are likewise preferred non-ionic polymers;
cellulose ethers, such as hydroxypropyl cellulose, hydroxyethyl cellulose and methylhydroxypropyl cellulose, such as those sold under the trademarks Culminal® and Benecel® (AQUALON), and Natrosol® types (Hercules);
starch and derivatives thereof, in particular starch ethers, for example, Structure® XL (National Starch), a multifunctional, salt-tolerant starch;
shellac;
polyvinyl pyrrolidones such as those sold under the name Luviskol® (BASF);
siloxanes. The siloxanes can be both water-soluble and water-insoluble. Both volatile and non-volatile siloxanes are suitable, wherein compounds whose boiling point under normal pressure is above 200° C. are understood to be non-volatile siloxanes. Preferred siloxanes are polydialkyl siloxanes, such as polydimethyl siloxane, polyalkylaryl siloxanes, such as polyphenylmethyl siloxane, ethoxylated polydialkyl siloxanes, as well as polydialkyl siloxanes containing amine and/or hydroxyl groups;
glycoside-substituted silicones.

It is also possible for the preparations to contain a plurality of, particularly two different polymers of the same charge and/or an ionic and an amphoteric and/or non-ionic polymer.

The additional polymers (G) are preferably present in agents according to the invention in amounts from 0.05 to 10 wt. %, based on total agent. Amounts of 0.1 to 5, in particular 0.1 to 3 wt. %, are particularly preferred.

Silicones are a particularly preferred group of ingredients.

Preferred agents according to the invention contain at least one silicone, preferably a silicone chosen from—
i) polyalkylsiloxanes, polyarylsiloxanes, polyalkylarylsiloxanes, which are volatile or non-volatile, straight-chain, branched or cyclic, crosslinked or non-crosslinked;
ii) polysiloxanes which in their general structure contain one or more organofunctional groups, which are selected from: substituted or unsubstituted aminated groups; (per)fluorinated groups; thiol groups; carboxylate groups; hydroxylated groups; alkoxylated groups; acyloxyalkyl groups; amphoteric groups; bisulfite groups; hydroxyacyl amino groups; carboxyl groups; sulfonic acid groups; and sulfate or thiosulfate groups;
iii) linear polysiloxane(A)-polyoxyalkylene(B) block copolymers of the type (A-B)ₙ, where n>3;
iv) grafted silicone polymers having a non-silicone-containing, organic framework consisting of an organic main chain formed from organic monomers containing no silicone, onto which at least one polysiloxane macromer has been grafted in the chain and optionally on at least one chain end;
v) grafted silicone polymers having a polysiloxane framework, onto which non-silicone-containing, organic monomers have been grafted, which have a polysiloxane main chain onto which at least one organic macromer containing no silicone has been grafted in the chain and optionally on at least one of its ends;
   or mixtures thereof.

Particularly preferred agents contain silicone(s) preferably in amounts from 0.1 to 10 wt. %, more preferably 0.25 to 7 wt. %, and in particular 0.5 to 5 wt. %, based on total weight of the agent. Preferred silicones are described below. Particularly preferred agents according to the invention contain at least one silicone of formula Si-I

(CH₃)₃Si—[O—Si(CH₃)₂]ₓ—O—Si(CH₃)₃ (Si-I),

wherein x is a number from 0 to 100, preferably 0 to 50, more preferably 0 to 20 and in particular 0 to 10. Under INCI nomenclature these silicones are known as DIMETHICONES.

Preferred silicones for use according to the invention have viscosities at 20° C. of 0.2 to 2 mm²s⁻¹, silicones having viscosities of 0.5 to 1 mm²s⁻¹ being particularly preferred.

Particularly preferred agents contain one or more amino-functional silicones. Such silicones can be described, for example, by the formula

M(RₐQ_{b}SiO_{(4-a-b)/2)x}(R_{c}SiO_{(4-c)/2)y}M

wherein R is a hydrocarbon or a hydrocarbon residue having 1 to approximately 6 carbon atoms, Q is a polar residue of the general formula —R¹HZ, wherein R¹ is a divalent linking group which is bonded to hydrogen and Z, composed of carbon and hydrogen atoms, carbon, hydrogen and oxygen atoms or carbon, hydrogen and nitrogen atoms, and Z is an organic amino-functional residue containing at least one amino-functional group; “a” is a value from approximately 0 to approximately 2, “b” is a value from approximately 1 to approximately 3, “a”+“b” is less than or equal to 3, and “c” is a number from approximately 1 to approximately 3, and x is a number from 1 to approximately 2000, preferably from approximately 3 to approximately 50 and most preferably from approximately 3 to approximately 25, and y is a number from approximately 20 to approximately 10,000, preferably from approximately 125 to approximately 10,000 and most preferably from approximately 150 to approximately 1000, and M is a suitable silicone end group, as is known in the prior art, preferably trimethylsiloxy. Non-restricting examples of R include alkyl residues such as methyl, ethyl, propyl, isopropyl, isopropyl, butyl, isobutyl, amyl, isoamyl, hexyl, isohexyl and similar; alkenyl residues, such as vinyl, halovinyl, alkyl vinyl, allyl, haloallyl, alkyl allyl; cycloalkyl residues, such as cyclobutyl, cyclopentyl, cyclohexyl and similar; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and similar, and sulfur-containing residues, such as mercaptoethyl, mercaptopropyl, mercaptohexyl, mercaptophenyl and similar. R is preferably an alkyl residue containing 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional residue containing at least one functional amino group. A possible formula for Z is NH(CH₂)_{z}NH₂, wherein z is 1 or more. Another possible formula for Z is —NH(CH₂)_{z}(CH₂)_{zz}NH, wherein both z and zz are independently 1 or more, this structure encompassing diamino ring structures such as piperazinyl. Z is most preferably an —NHCH₂CH₂NH₂ residue. Another possible formula for Z is —N(CH₂)_{z}(CH₂)_{zz}NX₂ or —NX₂, wherein each X of X₂ is independently chosen from the group consisting of hydrogen and alkyl groups having 1 to 12 carbon atoms, and zz is 0.

Q is most preferably a polar, amine-functional residue of the formula —CH₂CH₂CH₂NHCH₂CH₂NH₂. In the formulae “a” is a value from approximately 0 to approximately 2, “b” is a value from approximately 2 to approximately 3, “a”+“b” is less than or equal to 3, and “c” is a number from approximately 1 to approximately 3. The molar ratio of the RₐQ_{b}SiO_{(4-a-b)/2} units to the R_{c}SiO_{(4-c)/2} units is from approximately 1:2 to 1:65, preferably from approximately 1:5 to approximately 1:65 and most preferably from approximately 1:15 to approximately 1:20. If one or more silicones of the above formula is used, then the different variable substituents in the above formula can differ in the different silicone components that are present in the silicone mixture.

Preferred agents according to the invention contain an amino-functional silicone of formula (Si-II)

R′ₐR₃₋ₐ—Si(OSiG₂)ₙ-(OSiG_{b}R′_{2-b})ₘ—O—SiG₃₋ₐ-R′ₐ (Si-II),

wherein
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number from 0 to 3, in particular 0;
b is a number from 0 to 1, in particular 1;
m and n are numbers whose sum (m+n) is from 1 to 2000, preferably 50 to 150, wherein n preferably assumes values from 0 to 1999 and in particular from 49 to 149, and m preferably assumes values from 1 to 2000, in particular from 1 to 10,
R′ is a monovalent residue chosen from -Q—N(R″)—CH₂—CH₂—N(R″)₂, -Q—N(R″)₂, -Q—N⁺(R″)₃A⁻, -Q—N⁺H(R″)₂A⁻, -Q—N⁺H₂(R″)A⁻, -Q—N(R″)—CH₂—CH₂—N⁺R″H₂A⁻,
wherein each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—, and
R″ is identical or different residues from the group comprising —H, -phenyl, -benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

Particularly preferred agents according to the invention contain at least one amino-functional silicone of formula (Si-IIa)

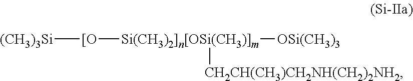

wherein m and n are numbers whose sum (m+n) is from 1 to 2000, preferably from 50 to 150, wherein n preferably assumes values from 0 to 1999 and in particular from 49 to 149, and m preferably assumes values from 1 to 2000, in particular from 1 to 10.

Under INCI declaration these silicones are known as Trimethylsilylamodimethicone.

Agents according to the invention are also particularly preferred which contain an amino-functional silicone of the formula (Si-IIb)

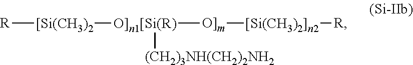

wherein R is —OH, —O—CH₃ or a —CH₃ group and m, n1 and n2 are numbers whose sum (m+n1+n2) is from 1 to 2000, preferably 50 to 150, wherein the sum (n1+n2) preferably assumes values from 0 to 1999 and in particular from 49 to 149, and m preferably assumes values from 1 to 2000, in particular from 1 to 10.

Under INCI declaration these silicones are known as Amodimethicone.

Regardless of which amino-functional silicones are used, preferred agents according to the invention contain an amino-functional silicone whose amine value is 0.25 meq/g or greater, preferably 0.3 meq/g or greater, and in particular 0.4 meq/g or greater. The amine value is the milli-equivalents of amine per gram of the amino-functional silicone. It can be determined by titration and also specified in the unit mg KOH/g.

Agents that are preferred according to the invention contain, relative to their weight, 0.01 to 10 wt. %, preferably 0.1 to 8 wt. %, more preferably 0.25 to 7.5 wt. % and in particular 0.5 to 5 wt. % of amino-functional silicone(s).

Cyclic dimethicones referred to under INCI as CYCLOMETHICONE can also be used to advantage according to the invention. Agents according to the invention are preferred here which contain at least one silicone of the formula Si-III

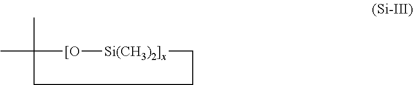

wherein x is a number from 3 to 200, preferably from 3 to 10, more preferably from 3 to 7 and in particular 3, 4, 5 or 6.

The silicones described above have a backbone which is made up of —Si—O—Si units. These Si—O—Si units can of course also be interrupted by carbon chains. Corresponding molecules can be obtained by chain extension reactions and are preferably used in the form of silicone-in-water emulsions.

Agents that are likewise preferred according to the invention contain at least one silicone of formula Si-IV

R₂Si—[O—SiR₂]ₓ—(CH₂)ₙ—[O—SiR₂]_{y}—O—SiR₃ (Si-IV),

wherein R is identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y are a number from 0 to 200, preferably from 0 to 10, more preferably from 0 to 7 and in particular 0, 1, 2, 3, 4, 5 or 6, and n is a number from 0 to 10, preferably from 1 to 8 and in particular 2, 3, 4, 5, 6.

The silicones are preferably water-soluble. Agents that are preferred according to the invention contain at least one water-soluble silicone.

For aesthetic reasons, “clear” products are often preferred by consumers. Hair treatment agents that are preferred according to the invention therefore are transparent or translucent. Transparent or translucent within the context of the present invention refers to a composition having an NTU value of below 100. The NTU unit (nephelometric turbidity unit) is a unit for turbidity measurements in liquids that is used in water treatment. It is the unit for a turbidity of a liquid measured with a calibrated nephelometer.

Agents according to the invention have advantageous properties and likewise impart advantageous properties to hair that is treated with them. Advantages were observed in particular in the treatment of the hair and scalp. Hair treatment agents according to the invention thus increase the elasticity of hair treated with them and lead to a strengthening of the internal structure of the hair fibers, reflected, for example, in higher melt temperatures in differential thermal analysis.

There is also an improvement in wet and dry combability and a prevention of premature formation of split ends in treated hair. On the skin and in particular on the scalp, the agents provide an increase in elasticity and, surprisingly, sebum-regulating effects. This prevents or lessens the visual impression of “greasy” skin or hair.

As has already been mentioned, compositions according to the invention are preferably hair treatment agents, in particular styling agents. Compositions that are particularly preferred are formulated as a styling gel, a pump-action hair spray, an aerosol hair spray, a pump-action hair foam or an aerosol hair foam.

The present invention also provides a method for temporarily shaping keratinic fibers, wherein a cosmetic composition according to the invention is applied to the hair as a pump-action hair spray, aerosol hair spray, pump-action hair foam, aerosol hair foam or styling gel and optionally worked into the hair with the palms of the hands and/or the fingers.

As mentioned above, composition used in this method preferably has a weakly acid to acid pH. The ammonium group can then be converted into an amino group by post-treatment with a neutral to weakly alkaline composition, making O->N acyl transfer possible, which allows a long-lasting fixing to keratinic fibers.

Preferred methods according to the invention therefore include, after working the composition according to the invention into the hair, treating the hair with a further composition having a pH of 6.5 to 11, preferably 7 to 10.5, more preferably 7.5 to 10.5 and in particular 8 to 9.5.

The composition is preferably then rinsed out of the hair. Methods according to the invention are preferred here wherein, after a contact period of 30 to 1200 seconds, preferably 60 to 900 seconds, particularly preferably 120 to 750 seconds and in particular 180 to 600 seconds, the further composition is rinsed out of the hair.

All that has been stated in respect of the agents according to the invention applies with necessary alterations to further preferred embodiments of the method according to the invention.

We claim:

1. Cosmetic composition comprising, in a cosmetically acceptable carrier:
at least one substance having at least one structural unit according to general formula (I) 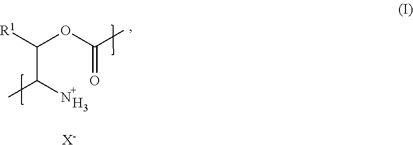
wherein R¹ is an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 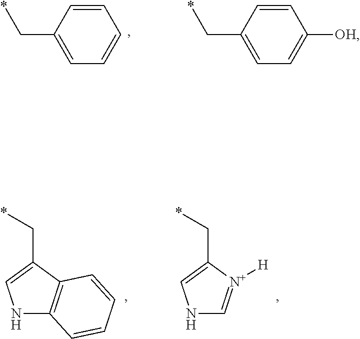
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 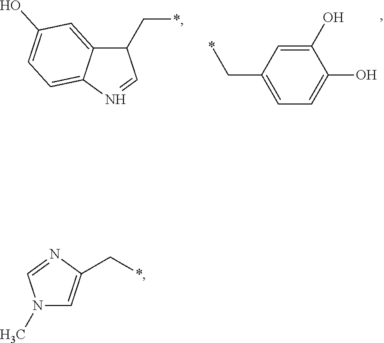
and
X⁻ is a physiologically tolerable anion or the m^{th} part of an m-times charged ion.

2. Composition according to claim 1, further comprising at least one substance having at least one structural unit according to general formula (I-a) 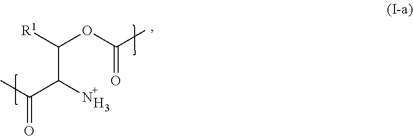
wherein R¹ and X⁻ are defined as above.

3. Composition according to claim 1, further comprising at least one substance having at least one structural unit according to general formula (I-b) 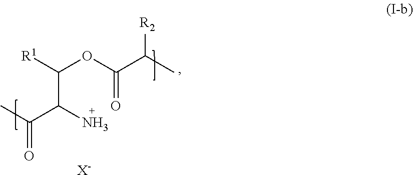
wherein R¹ and R² are each independently an H atom or —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 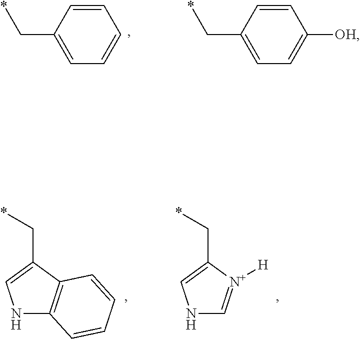
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 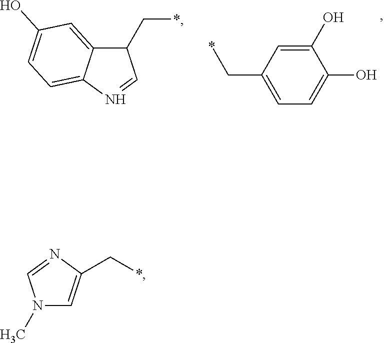
and
X⁻ is a physiologically tolerable anion or the m^{th} part of an m-times charged anion, preferably chloride, bromide, iodide, ½ sulfate, ⅓ citrate, ⅓ phosphate, methosulfate, p-toluenesulfonate.

4. Composition according to claim 3, further comprising at least one substance having at least one structural unit of general formula (I-c) 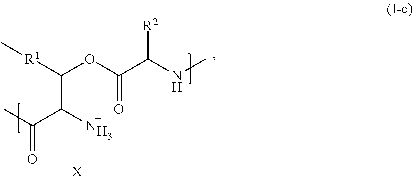
wherein R¹, R² and X⁻ are defined as above.

5. Composition according to claim 1, further comprising at least one substance having at least one structural unit of the general formula (I-d) 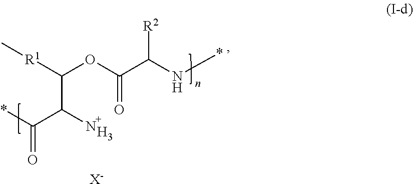
wherein R¹, R² and X⁻ are defined as above and n is a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40.

6. Composition according to claim 1, further comprising at least one substance of the formula (II) 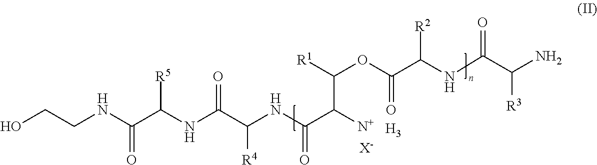
wherein R¹, R², n and X⁻ are defined as above, and R³, R⁴ and R⁵ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 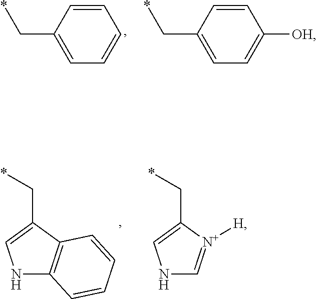
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂,

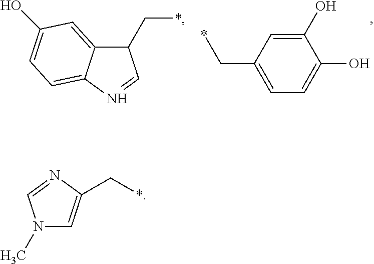

7. Composition according to claim 1, further comprising at least one substance of formula (III) 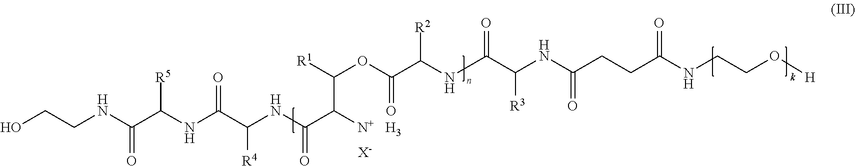
wherein R¹, R², n and X⁻ are defined as above, and R³, R⁴ and R⁵ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 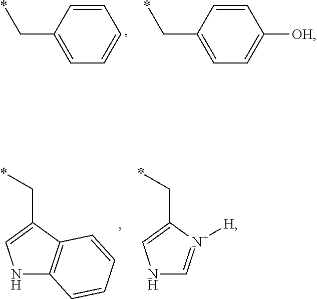
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 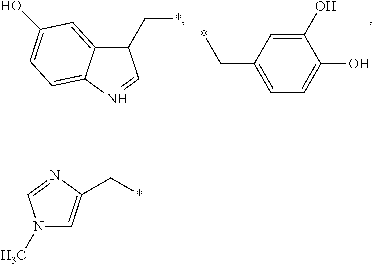
and k is a value from 1 to 100.

8. Composition according to claim 1, further comprising at least one substance of formula (IV) 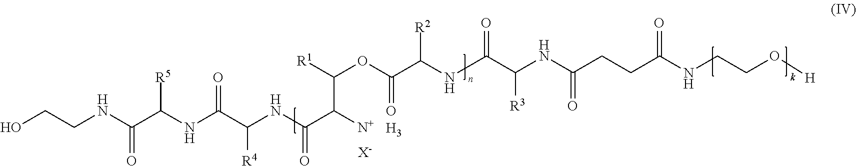
wherein R¹, R², n and X⁻ are defined as above, and R³, R⁴ and R⁵ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 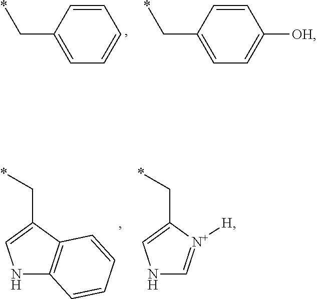
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 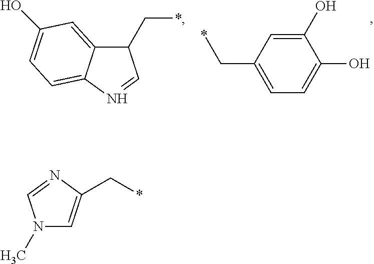
and k denotes values from 1 to 50, preferably 1, 2, 3, 4, 5, 6, 7, 8, 9 or 10.

9. Composition according to claim 1, further comprising at least one substance of formula (VI) 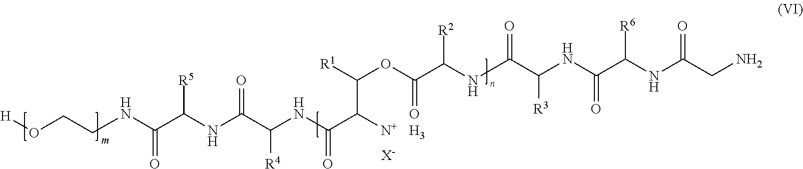
wherein R¹, R², n and X⁻ are defined as above, and R³, R⁴, R⁵ and R⁶ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 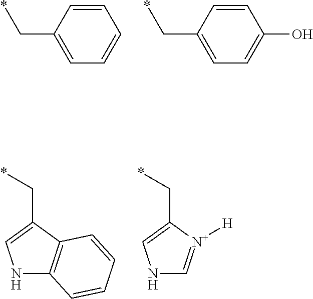
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 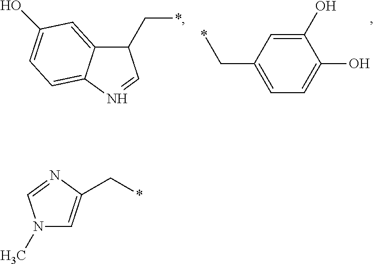
and m is a value from 1 to 50.

10. Composition according to claim 1, further comprising at least one substance of formula (VI) 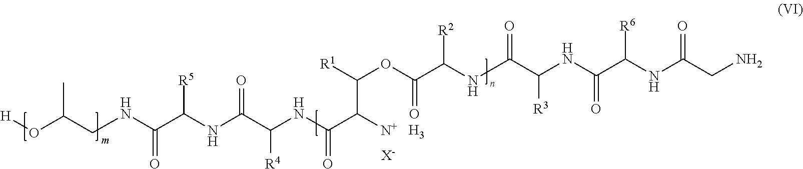
wherein R¹, R², n and X⁻ are defined as above, and R³, R⁴, R⁵ and R⁶ are independently chosen from —H, —CH₃, —CH(CH₃)₂, —CH₂CH(CH₃)₂, —CH(CH₃)CH₂CH₃, —CH₂—COOH, —CH₂CH₂—COOH, —CH₂—CO(NH₂), —CH₂CH₂—CO(NH₂), CH₂OH, —CH(OH)CH₃, —CH₂SH, —CH₂CH₂—S—CH₃, —(CH₂)₄—N⁺H₃, —(CH₂)₃—NH—C═N⁺H₂(NH₂), 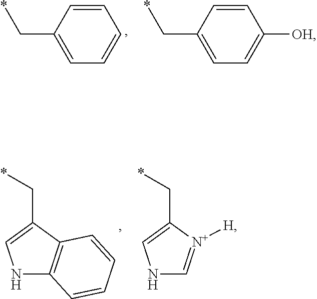
—CH₂—S—S—CH₂—CH(NH₂)COOH, —(CH₂)₃NH—C(O)NH₂, —CH₂CH₂C(O)NH(CH₂CH₃), —CH₂CH₂—SH, —CH₂—S(O)—CH₂—CH═CH₂, —CH₂—OPO₃H₂, —CH₂CH₂CH₂NH₂, 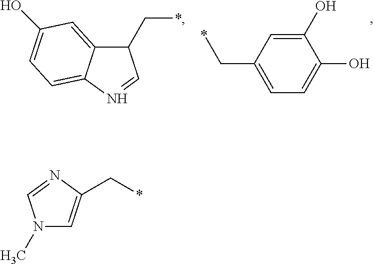
and m is a value from 1 to 100.

11. Composition according to claim 1, wherein the substance(s) containing at least one structural unit of general formula (I) have molar masses of 250 to 100,000 gmol⁻¹.

12. Composition according to claim 1, wherein the at least one structural unit of the general formula (I) is present in an amount of 0.001 to 10 wt. %, based on total weight of the agent.

13. Method for temporarily shaping keratinic fibers comprising:
applying a cosmetic composition according to claim 1 to the hair, and
optionally working the composition into the hair with the palms of the hands and/or the fingers.

14. Method according to claim 13, wherein the cosmetic composition is applied as a pump-action hair spray, aerosol hair spray, pump-action hair foam, aerosol hair foam or styling gel.

15. Method according to claim 13, further comprising treating the hair with a further composition having a pH of 6.5 to 11 after working the composition into the hair.

16. Method according to claim 14, further comprising rinsing the further composition out of the hair after a contact period of 30 to 1200 seconds.
